SEQUENCE LISTING (26 sequences)

SEQ ID NO: 1 - DNA, 214 bp, Homo sapiens
tgtcaacatagtgtgacgggaaggagcatgatgagacagaaggaaggtttaaactgggaaatctgagaaatggtatggttgtatgtgggttggcattcttgcatgatgggagtggccacctgctttcatattctgaagtcagagtgttccagacagaagaaatagcaagtgccgagaagctggcatcagaaaaacagaggggagatttgtgtgg

SEQ ID NO: 2 - DNA, 2497 bp, Homo sapiens
aaatctagctgatatagtgtggctcaaaaccttcagcacaaatcacaccgttagactatctggtgtggcccaaaccttcaggtgaacaaaggcactctaatctggcaggatattccaaagcattagagatgacctcttgcaaagaaaaagaaatggaaaagaaaaagaaagaaaggaaaaaaaaaaaaaaaaagagatgacctctcaggctctgaggggaaacgcctgaggtctttgagcaaggtcagtcctctgttgcacagtctccctcacagggtcattgtgacgatcaaatgtggtcacgtgtatgaggcaccaacacatgcctggctctggggagtgccgtgtaagtgtatgcttgcactgctgaatggctgggatgtgtcagggattatcttcagcacttacagatgctcatctcatcctcacagcatcactatgggatgggtattactggcctcatttgatggagaaagtggctgtggctcagaaaggggggaccactagaccagggacactctggatgctggggactccagagaccatgaccactcaccaactgcagagaaattaattgtggcctgatgtccctgtcctggagagggtggaggtggaccttcactaacctcctaccttgaccctctcttttagggctctttctgacctccaccatgatactaggaccccattgtattctgtaccctcttgactctatgacccccactgcccactgcatccagctgggtcccctcctatctctattcccagctggccagtgcagtctcagtgcccacctgtttgtcagtaactctgaaggggctgacattttactgacttgcaaacaaataagctaactttccagagttttgtgaatgctggcagagtccatgagactcctgagtcagaggcaaaggcttttactgctcacagcttagcagacagcatgaggttcatgttcacattagtacaccttgcccccccccaaatcttgtagggtgaccagagcagtctaggtggatgctgtgcacacggggtttgtgccactggtgaggaacctgagattaggaatcctcaatcttatactgggacaacttgcaaacctgctcagcctttgtctctgatgaagatattatcttcatgatcttggattgaaaacagacctactctggaggaacatattgtattgattgtccttgacagtaaacaaatctgttgtaagagacattatctttattatctaggacagtaagcaagcctggatctgagagagatatcatcttgcaaggatgcctgctttacaaacatccttgaaacaacaatccagaaaaaaaggtgttgctgtctttgctcagaagacacacagatacgtgacagaaccatggtaaccagagcagtctaggtggatgctgtgcacacggggtttgtgccactggtgaggaacctgagattaggaatcctcaatcttatactgggacaacttgcaaacctgctcagcctttgtctctgatgaagatattatcttcatgatcttggattgaaaacagacctactctggaggaacatattgtattgattgtccttgacagtaaacaaatctgttgtaagagacattatctttattatctaggacagtaagcaagcctggatctgagagagatatcatcttgcaaggatgcctgctttacaaacatccttgaaacaacaatccagaaaaaaaggtgttgctgtctttgctcagaagacacacagatacgtgacagaaccatggagaattgcctcccaacactgttcagccagagccttccacccttgtctgcaggacagtctcaacgttccaccattaaatacttcttctgtcacatcctgcttacttatgcctaaccaaggttctaggtcccgatcgactgtgtctggcagcactccactgccaaacccagaataaggcagcgctcaggatctttttatgatgacagtagcaatgtatctgtggagctggattctgggttgggagtgcaaggaaaagaatgtactaaatgccaagacatctatttcaggagcatgaggaataaaagttctagtttctggtctcagagtggtgcagggatcagggagtctcacagtctcctgagtgctggtgtcttagggcacactgggtcttggagtgcaaaggatctaggcacgtgaggctttgtatgaagaatcggggatcgtacccaccccctgtttctgtttcatcctgggcgtgtctcctctgcctttgtcccctagatgaagtctccatgagccacagggcctggtgcatccagggtgatctagtaattgcagaacagcaagtactagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagcc

SEQ ID NO: 3 - DNA, 259 bp, Bos taurus
gctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagacggaaagaac

SEQ ID NO: 4 - DNA, 1053 bp, Bacteriophage P1
atgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 5 - DNA, 133 bp, Artificial Sequence
  5′ -donor site from the first intron of the human B-globin and the branch and 3′ - acceptor site from the intron located between the leader and body of an immunoglobulin gene heavy chain variable region
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 6 - DNA, 41316 bp, Artificial Sequence
  PCA3 is from Homo sapiens, CRE is from Bateriophage P1 with a chimeric intron, PSEBC is a modified version os the PSA promoter from Homo sapiens and TSTA is an artificial construct
  misc_feature (31858)..(31858): n is a, c, g, or t
ttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagcaggtcgacaaatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatttaaaatacacaaacaattagaatcagtagtttaacacattatacacttaaaaattttatatttaccttagagctttaaatctctgtaggtagtttgtccaattatgtcacaccacagaagtaaggttccttcacaaagatcccaagctgtcgatcgacatttctagaggatctcggacccggggaatccccgtcccccaacatgtccagatcgaaatcgtctagcgcgtcggcatgcgccatcgccacgtcctcgccgtctaagtggagctcgtcccccaggctgacatcggtcgggggggcggatctcggacccggggaatccccgtcccccaacatgtccagatcgaaatcgtctagcgcgtcggcatgcgccatcgccacgtcctcgccgtctaagtggagctcgtcccccaggctgacatcggtcgggggggcggatcccccgggctgcaggaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatggtggcgcgccataacttcgtataatgtatgctatacgaagttatcagctggttctttccgtctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagccgtacgataacttcgtataatgtatgctatacgaagttattgcgcaggctggggagcctcccccaggagccctataaaaccttcattccccaggactccgcccctgccctgctggcacccagaggctgaccaaggccctccccatgctgctggaggctggacaaccccctcccacacccagagctgtggaaggggagggagagctagtacttgctgttctgcaattactagatcaccctggatgcaccaggccctgtggctcatggagacttcatctaggggacaaaggcagaggagacacgcccaggatgaaacagaaacagggggtgggtacgatccccgattcttcatacaaagcctcacgtgcctagatcctttgcactccaagacccagtgtgccctaagacaccagcactcaggagactgtgagactccctgatccctgcaccactctgagaccagaaactagaacttttattcctcatgctcctgaaatagatgtcttggcatttagtacattcttttccttgcactcccaacccagaatccagctccacagatacattgctactgtcatcataaaaagatcctgagcgctgccttattctgggtttggcagtggagtgctgccagacacagtcgatcgggacctagaaccttggttaggcataagtaagcaggatgtgacagaagaagtatttaatggtggaacgttgagactgtcctgcagacaagggtggaaggctctggctgaacagtgttgggaggcaattctccatggttctgtcacgtatctgtgtgtcttctgagcaaagacagcaacacctttttttctggattgttgtttcaaggatgtttgtaaagcaggcatccttgcaagatgatatctctctcagatccaggcttgcttactgtcctagataataaagataatgtctcttacaacagatttgtttactgtcaaggacaatcaatacaatatgttcctccagagtaggtctgttttcaatccaagatcatgaagataatatcttcatcagagacaaaggctgagcaggtttgcaagttgtcccagtataagattgaggattcctaatctcaggttcctcaccagtggcacaaaccccgtgtgcacagcatccacctagactgctctggttaccatggttctgtcacgtatctgtgtgtcttctgagcaaagacagcaacacctttttttctggattgttgtttcaaggatgtttgtaaagcaggcatccttgcaagatgatatctctctcagatccaggcttgcttactgtcctagataataaagataatgtctcttacaacagatttgtttactgtcaaggacaatcaatacaatatgttcctccagagtaggtctgttttcaatccaagatcatgaagataatatcttcatcagagacaaaggctgagcaggtttgcaagttgtcccagtataagattgaggattcctaatctcaggttcctcaccagtggcacaaaccccgtgtgcacagcatccacctagactgctctggtcaccctacaagatttgggggggggcaaggtgtactaatgtgaacatgaacctcatgctgtctgctaagctgtgagcagtaaaagcctttgcctctgactcaggagtctcatggactctgccagcattcacaaaactctggaaagttagcttatttgtttgcaagtcagtaaaatgtcagccccttcagagttactgacaaacaggtgggcactgagactgcactggccagctgggaatagagataggaggggacccagctggatgcagtgggcagtgggggtcatagagtcaagagggtacagaatacaatggggtcctagtatcatggtggaggtcagaaagagccctaaaagagagggtcaaggtaggaggttagtgaaggtccacctccaccctctccaggacagggacatcaggccacaattaatttctctgcagttggtgagtggtcatggtctctggagtccccagcatccagagtgtccctggtctagtggtcccccctttctgagccacagccactttctccatcaaatgaggccagtaatacccatcccatagtgatgctgtgaggatgagatgagcatctgtaagtgctgaagataatccctgacacatcccagccattcagcagtgcaagcatacacttacacggcactccccagagccaggcatgtgttggtgcctcatacacgtgaccacatttgatcgtcacaatgaccctgtgagggagactgtgcaacagaggactgaccttgctcaaagacctcaggcgtttcccctcagagcctgagaggtcatctctttttttttttttttttcctttctttctttttcttttccatttctttttctttgcaagaggtcatctctaatgctttggaatatcctgccagattagagtgcctttgttcacctgaaggtttgggccacaccagatagtctaacggtgtgatttgtgctgaaggttttgagccacactatatcagctagatttatttaaattcgacggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactctagaattacacggcgatctttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgcgacacctgcgtcgaagatgttggggtgttggagcaagatggattccaattcagcgggagccacctgatagcctttgtacttaatcagagacttcaggcggtcaacgatgaagaagtgttcgtcttcgtcccagtaagctatgtctccagaatgtagccatccatccttgtcaatcaaggcgttggtcgcttccggattgtttacataaccggacataatcataggacctctcacacacagttcgcctctttgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgcctgatacctggcagatggaacctcttggcaaccgcttccccgacttccttagagaggggagcgccaccagaagcaatttcgtgtaaattagataaatcgtatttgtcaatcagagtgcttttggcgaagaaggagaatagggttggcaccagcagcgcactttgaatcttgtaatcctgaaggctcctcagaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctcacgcaggcagttctatgaggcagagcgacacctttaggcagaccagtagatccagaggagttcatgatcagtgcaattgtcttgtccctatcgaaggactctggcacaaaatcgtattcattaaaaccgggaggtagatgagatgtgacgaacgtgtacatcgactgaaatccctggtaatccgttttagaatccatgataataattttttggatgattgggagctttttttgcacgttcaaaattttttgcaacccctttttggaaacgaacaccacggtaggctgcgaaatgcccatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtactcagcgtaagtgatgtccacctcgatatgtgcatctgtaaaagcaattgttccaggaaccagggcgtatctcttcatagccttatgcagttgctctccagcggttccatcttccagcggatagaatggcgccgggcctttctttatgtttttggcgtcttccatggtggctttaccaacagtaccggaatgccaagcttacttagatcgcagatctcgagacaccactcgacacggcaccagctcaatcagtcacagtgtaaaaaagggccaagtgcagagcgagtatatataggactggggatcctctagagtcggagtactgtcctccgagcggagtactgtcctccgagcggagtactgtcctccgagcggagtactgtcctccgagcggagtactgtcctccgaggacctgcaggcatgcaagcttgtattctatagtgtcacctaaatgagctcggtaccacaactttgtatacaaaagttgggtacccctcgagtgtcaacatagtgtgacgggaaggagcatgatgagacagaaggaaggtttaaactgggaaatctgagaaatggtatggttgtatgtgggttggcattcttgcatgatgggagtggccacctgctttcatattctgaagtcagagtgttccagacagaagaaatagcaagtgccgagaagctggcatcagaaaaacagaggggagatttgtgtgggtcgaccaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggtgtaagtatcaaggttacaagacaggtttaaggagaccaatagaaactgggcttgtcgagacagagaagactcttgcgtttctgataggcacctattggtcttactgacatccactttgcctttctctccacagtcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgctataattatttgatatttatggtgacatatgagaaaggatttcaacatcgacggaaaatatgtagtgctgtctgtaagcactaatattcagtcgccagccgtcattgtcactgtaaagctgagcgatagaatgcctgatattgactcaatatccgttgcgtttcctgtcaaaagtatgcgtagtgctgaacatttcgtgatgaatgccaccgaggaagaagcacggcgcggttttgctaaagtgatgtctgagtttggcgaactcttgggtaaggttggaattgtcgaggctgggtgtggcggacaagcttgtattctatagtgtcacctaaatcccagctttacccagctttc

SEQ ID NO: 7 - DNA, 485 bp, Homo sapiens
tgactcatgactcatgactcagaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 8 - DNA, 465 bp, Homo sapiens
agaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 9 - DNA, 42 bp, Artificial Sequence
  DNA Primer
ggatccgtcgaatttaaataaatctagctgatatagtgtggc

SEQ ID NO: 10 - DNA, 38 bp, Artificial Sequence
  DNA primer
atacgaagttattgcgcaggctggggagcctcccccag

SEQ ID NO: 11 - DNA, 37 bp, Artificial Sequence
  DNA primer
ggggagatttgtgtgggtcgacaccatgcccaagaag

SEQ ID NO: 12 - DNA, 40 bp, Artificial Sequence
  DNA primer
gtaaccttgatacttacacctggtcgaaatcagtgcgttc

SEQ ID NO: 13 - DNA, 23 bp, Artificial Sequence
  DNA primer
tcgttcactcatggaaaatagcg

SEQ ID NO: 14 - DNA, 39 bp, Artificial Sequence
  DNA primer
gtacaagaaagctgggtaaagcttgtccgccacacccag

SEQ ID NO: 15 - DNA, 31 bp, Artificial Sequence
  DNA primer
gtaagtatcaaggttacaagacaggtttaag

SEQ ID NO: 16 - DNA, 43 bp, Artificial Sequence
  DNA primer
ctattttccatgagtgaacgactgtggagagaaaggcaaagtg

SEQ ID NO: 17 - DNA, 18 bp, Artificial Sequence
  DNA primer
aagtagagggctgggctg

SEQ ID NO: 18 - DNA, 36 bp, Artificial Sequence
  DNA primer
ccatgagtgaacgaacctagaaaggtaagaaaagtg

SEQ ID NO: 19 - DNA, 19 bp, Artificial Sequence
  DNA primer
gtagagggctgggctgggc

SEQ ID NO: 20 - DNA, 34 bp, Artificial Sequence
  DNA primer
ccatgagtgaacgactagaaaggtaagaaaagtg

SEQ ID NO: 21 - DNA, 21 bp, Artificial Sequence
  DNA primer
tacggctgatcagcctcgact

SEQ ID NO: 22 - DNA, 26 bp, Artificial Sequence
  DNA primer
aatgacacctactcagacaatgcgat

SEQ ID NO: 23 - DNA, 25 bp, Artificial Sequence
  DNA probe
tgcccctcccccgtgccttccttga

SEQ ID NO: 24 - DNA, 24 bp, Artificial Sequence
  DNA primer
atggtggctttaccaacagtaccg

SEQ ID NO: 25 - DNA, 25 bp, Artificial Sequence
  DNA primer
tatgaagagatacgccctggttcct

SEQ ID NO: 26 - DNA, 27 bp, Artificial Sequence
  DNA probe
aggcccggcgccattctatccgctgga

(19) United States
(12) Patent Application Publication
(10) Pub. No.: US 2020/0140951 A9
(43) Pub. Date: May 7, 2020

(54) Transcriptional system and uses thereof for single cell detection

(71) Applicant: Université Laval, Québec (CA)

(72) Inventors: Frédéric Pouliot, Québec, CA (US); Bertrand Neveu, Québec, CA (US); Pallavi Jain, Québec, CA (US)

(21) Appl. No.: 15/957,672

(22) Filed: Apr. 19, 2018

Related U.S. Application Data
(60) Provisional application No. 62/487,293, filed on Apr. 19, 2017.

(30) Foreign Application Priority Data
Apr. 19, 2017 (CA) 2964605

(51) Int. Cl. C12Q 1/6886; C12Q 1/6897; G01N 33/50

(52) U.S. Cl. CPC C12Q 1/6886; C12Q 2600/136; G01N 33/5011; C12Q 1/6897

(57) ABSTRACT

The present invention relates to transcriptional systems and method of uses thereof for identifying and/or assessing cancer cells from biological fluid(s) of cancer patients.

### RELATED APPLICATIONS

This application claims priority under 35 U.S.C. § 119 or 365 to Canadian Application No. 2,964,605, filed Apr. 19, 2017, and claims the benefit of U.S. Provisional Application No. 62/487,293, filed Apr. 19, 2017. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 54931001002SequenceListing.txt; created Apr. 19, 2018, 64 KB in size.

### TECHNICAL FIELD

The present invention relates to the fields of cell biology, molecular biology, cancer biology, and medicine. More particularly, the present invention relates to transcriptional systems for detecting and assessing cancer cells.

### BACKGROUND

Currently, clinical therapies for patients rely on the recognition of the molecular drivers of the disease. Improvements in the clinical outcome of many cancer types is likely to be achieved by giving patients a drug tailored to the genetic make up of their tumor. Biomarkers predicting therapy response are frequently evaluated on tumor biopsy samples which incorporate bulk analysis of whole tissue samples. Therefore, response predictive biomarkers panels are built based on the presence of resistance or response genotypes rather than a percentage of responding over non-responding cells. Indeed, bulk tissue analysis may lead to partial response of a sensitive cell subpopulation while another clone progresses until clinical progression is detected and treatment changed. Similarly, some treatments might be discarded secondary to the detection of resistance genotypes in a minority of cells while the patient would have responded. The ratio of responsive over non-responsive cells may therefore key to direct patients towards best treatment.

For example, in metastatic castration resistant prostate cancer (mCRPC), several treatments have been shown to increase overall survival. Among them, enzalutamide (ENZ) and abiraterone, are targeting the androgen receptor axis pathway (SLAATT=second-line androgen axis targeting therapies)1. Among CRPC drug arsenal, SLAATT are key treatments because they increase patient's overall survival before or after chemotherapy. Despite efficacy, between 20 and 80% of patients will be resistant to SLAAT and molecular tools that enable identification of SLAATT-resistant from sensitive cell populations are needed. Despite huge advances in omics, few biomarkers have been validated for clinical use in part because biomarkers cannot address complex multigene interactions that may lead to SLAATT resistance mechanisms. Moreover, bulk tissue samples analyzed do contain heterogeneous cell populations and the detection of a response biomarker (e.g. mutation in a sample) might only reflect a minority of cells decreasing its predictive value (http://www.ncbi.nlm.nih.gov/pubmed/26383955). This has been described specifically in prostate cancer (PCa) after single cell sequence analysis where 60% of metastatic patients harbored more than one resistance gene, expressed in different single cells. While PCa single cell growth ex vivo would be the best integrative approach to interrogate single cell drug sensitivity, experimental and biological limitations have decreased greatly the interest for such a method.

Currently, clinical therapies for patients rely on the recognition of the molecular drivers of the disease. Improvements in the clinical outcome of many cancer types is likely to be achieved by giving patients a drug tailored to the genetic make up of their tumor. Biomarkers predicting therapy response are frequently evaluated on tumor biopsy samples which incorporate bulk analysis of whole tissue samples. Therefore, response predictive biomarkers panels are built based on the presence of resistance or response genotypes rather than a percentage of responding over non-responding cells. Indeed, bulk tissue analysis may lead to partial response of a sensitive cell subpopulation while another clone progresses until clinical progression is detected and treatment changed. Similarly, some treatments might be discarded secondary to the detection of resistance genotypes in a minority of cells while the patient would have responded. The ratio of responsive over non-responsive cells may therefore key to direct patients towards best treatment. The method presented herein, which is based on drug-target single cell imaging, has a unique ability to be highly integrative at molecular, cellular and cell population levels. It can detect molecular changes by monitoring the activity of AR in real-time. Through dynamic imaging of single cells AR activity upon antiandrogen exposure, this method can also integrate most antiandrogen resistance mechanisms (resistome) and their interactions together to escape from antiandrogen inhibitory effects. At cellular levels, our method could be used to image heterogeneous responses of AR positive cells to antiandrogens in order to determine patient's sensitivity to antiandrogens such as Enz. By including AR− cell imaging in the model, a number of static and dynamic cellular data upon antiandrogen exposure could be registered and serve as mathematical model to predict response to antiandrogen treatment.

With disease progression under treatment pressure, it has been shown that cell genotypes are heterogeneous and plastic. This has lead to the development of several non-invasive techniques that are used to study various markers involved in disease progression and treatment response from patient's tissues, either from biopsy or body fluids. For most cancers, blood is used as a fluid biopsy for cancer monitoring or prognosis. However, other sources of body fluid such as peritoneal (ascites), urine, pleural or cerebrospinal fluids have also been exploited to harvest cancer. Anatomical position of prostate cancers make urine an important source of cancer cells and methods to detect PCa cells from urine using cytological and multiplex immunofluorescence techniques have been reported but cannot identify living cells.

Therefore, there is a great need to develop new integrative approaches for treatment response prediction based on single cells.

### SUMMARY

The present description relates to a method for identifying one or more single cancer cells from cells harvested from a biological fluid of a patient, said method comprising:
providing a bioluminescent substrate;
providing a culture medium comprising one or more harvested cancer cells transduced with or comprising a construct comprising:
   a site specific recombinase under the control of a first system;
   a bioluminescent reporter gene under the control of a second system; and
   a site-specific recombination sequence, said site-specific recombination sequence located upstream of the bioluminescent reporter gene;
   wherein, upon activation of the first and second systems, said transduced cancer cells express the bioluminescent reporter gene following recombination;
   wherein at least one of the first and second systems is a response treatment system and the other system is a cancer specific promoter or a tissue specific promoter; and
imaging harvested cancer cells by bioluminescence microscopy to identify said one or more single cancer cells.

The present description relates to a method for dynamically assessing the response of one or more single cancer cells from cells harvested from a biological fluid of a patient to a treatment, said method comprising:
providing a bioluminescent substrate;
providing a culture medium comprising one or more harvested cancer cells transduced with or comprising a construct comprising:
   a site specific recombinase under the control of a first system;
   a bioluminescent reporter gene under the control of a second system; and
   a site-specific recombination sequence, said site-specific recombination sequence located upstream of the bioluminescent reporter gene;
   wherein, upon activation of the first and second systems, said transduced cancer cells express the bioluminescent reporter gene following recombination;
   wherein at least one of the first and second systems is a response treatment system and the other system is a cancer specific promoter or a tissue specific promoter; and
imaging harvested cancer cells by bioluminescence microscopy to get a baseline reading;
treating harvested cancer cells with a treatment;
imaging harvested cancer cells by bioluminescence microscopy to get a post-treatment reading; and
determining whether the one or more assessed cancer cells have responded to the treatment based on the difference between the baseline reading and the post-treatment reading.

The present description relates to a method for identifying one or more single prostate cancer cells from cells harvested from the urine of a patient, said method comprising:
providing a bioluminescent substrate;
providing a culture medium comprising one or more harvested cancer cells transduced with a construct comprising:
   a Cre recombinase under the control of a PCA3 promoter;
   a bioluminescent reporter gene under the control of a PSA promoter; and
   a Cre site-specific recombination sequence, said Cre site-specific recombination sequence located upstream of the bioluminescent reporter gene;
   wherein, upon activation of the PCA3 and PSA promoters, said transduced cancer cells express the bioluminescent reporter gene following recombination; and
imaging harvested prostate cancer cells by bioluminescence microscopy to identifying one or more single prostate cancer cells.

The present description relates to the use of a construct for identifying one or more single prostate cancer cells from cells harvested from the urine of a patient by bioluminescence microscopy, wherein said one or more single prostate cancer cells comprise a construct comprising:
a Cre recombinase under the control of a PCA3 promoter;
a bioluminescent reporter gene under the control of a PSA promoter; and
a Cre site-specific recombination sequence said Cre site-specific recombination sequence located upstream of the bioluminescent reporter gene;
wherein, upon activation of the PCA3 and PSA promoters, said cancer cells express the bioluminescent reporter gene following recombination.

The present description relates to the use of a method for dynamically assessing the antiandrogen response of one or more single prostate cancer cells from cells harvested from the urine of a patient, said method comprising:
providing a bioluminescent substrate; and
providing a culture medium comprising one or more harvested prostate cancer cells transduced with a construct comprising:
   a Cre recombinase under the control of a PCA3 promoter;
   a bioluminescent reporter gene under the control of a PSA promoter; and
   Cre site-specific recombination sequence, said Cre site-specific recombination sequence located upstream of the bioluminescent reporter gene;
   wherein, said transduced prostate cancer cells express the bioluminescent reporter gene following recombination upon activation of the PCA3 and PSA promoters;
imaging harvested prostate cancer cells by bioluminescence microscopy to get a baseline reading;
treating harvested prostate cancer cells with an antiandrogen treatment;
imaging harvested prostate cancer cells by bioluminescence microscopy to get a post-treatment reading; and
determining whether the one or more assessed cancer cells have responded to the antiandrogen treatment based on the difference between the baseline reading and the post-treatment readings.

The present description relates to the use of a construct for dynamically assessing the antiandrogen response of one or more single prostate cancer cells from cells harvested from the urine of a patient by bioluminescence microscopy, wherein said one or more single prostate cancer cells comprise a construct comprising:
a Cre recombinase under the control of a PCA3 promoter;
a bioluminescent reporter gene under the control of a PSA promoter; and
a Cre site-specific recombination sequence, said Cre site-specific recombination sequence located upstream of the bioluminescent reporter gene;
wherein, upon activation of the PCA3 and PSA promoters, said cancer cells express the bioluminescent reporter gene following recombination.

The present description relates to a construct as described herein, a biological sample (e.g. urine or blood) of a cancer patient comprising the construct as defined herein, or an isolated primary cancer cell transduced with a construct as defined herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 PCA3 promoter is PCa specific while PSEBC promoter is androgen responsive. (A) PCA3 promoter is highly PCa specific. (B) PSEBC promoter is active in androgen responsive cell lines. PCa cells (22Rv1, DU-145 and PC3, LNCaP-LN3, LNCaP and LNCaP-PRO5) and breast cancer cells (ZR-75, CAMA-1, MDA-MB-231 and MCF-7) were infected with a replication deficient adenovirus expressing the firefly luciferase gene under control of PCA3-TSTA or PSEBC-TSTA at 2 MOI. In case of PSEBC-TSTA, 24 hours post-infection media was changed to add DHT (10 nM). Seventy-two hours after infection the cells were lysed and luciferase activity was measured. The relative luciferase activity was first normalized by protein content in each well and then normalized according to the average of luciferase activity driven by SV40 promoter in each cell line (relative activity=(RLU/μg protein)÷(RLU SV40/μg). Each data represents triplicates±S.D. (C) Scheme for multi-promoter integrated TSTA (MP-ITSTA).

FIG. 2 Characterization of the MP-ITSTA system for prostate cancer-specific expression. (A) PSEBC-Cre-SV40-ITSTA could inhibit expression of SV40 promoter in androgen non-responsive prostate cancer cells (DU-145) and in non-PCa cells (SW-780) showing that reporter signal is expressed only when the two driving promoters are activated. Prostate Cancer (22Rv1, LAPC4 and DU-145), Bladder cancer (SW-780) and Breast cancer (CAMA-1) cells were infected with replication deficient adenovirus expressing the firefly luciferase gene under control of PSEBC-Cre-SV40-TSTA-fl or PSEBC-Cre-PCA3-TSTA-fl at 2 MOI. Twenty-hours later cells were treated with increasing concentrations of DHT (0-100 nM). Forty-eight hours later cells were lysed and luciferase assay was done. (B) PCA3-Cre-PSEBC-ITSTA could give a higher signal compared to PCA3-TSTA alone while being specific for PCa. 22Rv1 cells were infected with the PCA3-Cre-PSEBC-TSTA and PCA3-TSTA adenoviruses and were harvested at the indicated time points, and luciferase assay was done. (C) PCA3 dependent Cre expression leads to efficient deletion of DNA between the loxP sites. 22Rv1 cells were infected with replication deficient adenoviruses expressing the firefly luciferase gene under control of PCA3-Cre-PSEBC-TSTA-fl (with Cre) or PCA3-GFP-PSEBC-TSTA-fl (without Cre) at 2 MOI in the presence of DHT (10 nM). Cells were harvested at the indicated time points and viral DNA was isolated. Quantitative real-time PCR was done for the isolated DNA using two sets of primers indicated in the scheme: Primer set 1 amplifying a region within the stop cassette and Primer set 2 amplifying luciferase as internal control. Relative copy number (RCN)=((copy number of stop cassette/copy number of luciferase)/RCN at 6 hour×100). Relative luciferase activity was normalised over total protein and then normalized according to the average of luciferase activity driven by SV40 promoter in each cell line. (RLU)=((Firefly luciferase activity/total protein)/SV40 activity). Each data represents triplicates±SD.

FIG. 3 PCA3-Cre-PSEBC-ITSTA shows activity specifically in AR responsive PCa cells giving an induction comparable to PSEBC-TSTA with DHT. (A) Graph shows that fold induction seen with AR agonist DHT with PCA3-Cre-PSEBC-ITSTA is similar to PSEBC-TSTA. (B) Graph shows that PCA3-Cre-PSEBC-ITSTA is active only in AR sensitive prostate cancer cells. AR responsive (22Rv1, LAPC4, LNCaP), AR non-responsive (DU-145 and PC3) and AR responsive breast cancer (ZR-75 and CAMA-1) cells were infected with replication deficient adenovirus expressing the firefly luciferase gene under control of PSEBC-TSTA or PCA3-Cre-PSEBC-fl at 2 MOI. Twenty-four hours later media was changed to add DHT (10 nM). Forty-eight hours after treatment cells were lysed and luciferase activity was measured. The relative luciferase activity was first normalized by protein content in each well and then normalized according to the average of luciferase activity driven by SV40 promoter in each cell line (relative activity=(RLU/μg protein)÷(RLU SV40/μg) and represented as relative activity over 22Rv1 in the case of (A) or relative activity over bicalutamide in the case of (B). Each data represents triplicates±S.D.

FIG. 4 Bovine growth hormone poly A (BGHstop) could efficiently inhibit the expression of luciferase and gives better reactivation in the presence of Cre compared to SV40 poly A. (A) Schemes of the plasmids transfected. (B) Graph shows that BGHstop showed better expression than SV40stop. (C) Graph shows that increasing the concentration of Cre enhanced the expression of firefly luciferase. PCa cells (LAPC4) were co-transfected with plasmid pBS185 and pENTR-SV40-BGHstop-fl or pENTR-SV40-SV40stop-fl along with pGL3-renilla-null. Forty-eight hours after transfection the cells were lysed and luciferase assay was done. To assess the effect of increasing the expression of Cre protein multiple concentrations of pBS185 were used (100 ng-800 ng). Relative luciferase activity was normalised over renilla. RLU=(firefly luciferase activity/renilla null activity).

FIG. 5 Insertion of the chimeric human intron within Cre recombinase could inhibit leakage of Cre in bacteria and enhanced the expression of firefly luciferase. (A) Schemes of plasmid with intron in Cre gene. (B) Prostate cancer cells (LAPC4) were co-transfected with plasmids pENTR-PCA3-Cre (WT, BHG-Ig, Prm2 and Prm2-AG) and pENTR-SV40-stop-GAI4VP16-GAL4RE-fl along with pGL3-renilla-null. Seventy-two hours after transfection the cells were lysed and luciferase assay was done. Relative luciferase activity was normalised over renilla. RLU=(firefly luciferase activity/renilla null activity).

FIG. 6 Characterization of the best PCA3-Cre-PSEBC-TSTA conformations for prostate cancer-specific expression (A) Scheme of non-replicative reporter adenoviruses. (B) Amplification provided by orientation A was highest and had the least leakage expression. PCa cells (22Rv1, LAPC4, LNCaP, DU-145), breast cancer cells (CAMA-1) and bladder cancer cells (SW-780) were infected with non-replicative adenovirus with the above-mentioned orientations at 2 MOI. Seventy-two hours after infection the cells were lysed and luciferase assay was performed. Relative luciferase activity was normalised over total protein and then normalised over luciferase activity driven by SV40 promoter in each cell line. (relative activity=(RLU/μg protein)÷(RLU SV40/μg). Each data represents triplicates±SD.

FIG. 7 Optimization of a bioluminescence microscopy method for single cell imaging after adenoviral system transduction. (A) Scheme of the non-replicative adenoviruses used in the studies. (B) Amplification of the PSEBC-promoter signal by the Two-Step-Transcriptional Amplification system increases the number of AR− responsive PCa cells detected. LAPC4-GFP cells were transduced with 10⁴ infectious viral particles (ivp) of CMV-TSTA, PSEBC-TSTA or PSEBC-fl. Imaging was performed at 24, 48, and 72 h after D-luciferin addition and an exposure time of 5 min. (C) PSEBC-TSTA activity is more heterogeneous between single PCa cells when compared to a PCA3-promoter based imaging system (PCA3-3STA). 22Rv1-GFP cells were transduced using either PCA3-3STA or PSEBC-TSTA adenovirus and imaged after 72 h. The percentage of positive cells were analyzed as a ratio of luminescent over fluorescent cells. (D) Increasing the exposure time for imaging did not increase the number of cells detected after PSEBC-TSTA transduction. 22Rv1-GFP cells were transduced with PSEBC-TSTA. Seventy-two hours post-infection, imaging was performed at 20× magnification at exposure times of 5, 10, and 20 min. (E) Bioluminescence single cell microscopy is quantitative. Graph shows the linear increase in single cell (ROI 1-15) sum grey intensity over exposure time. (F) Representative images of 22Rv1 cells transduced with PSEBC-TSTA and plotted in (e) and showing that single cell luminescence increases with exposure time but not the number of detected cells. (G) Bioluminescence microscopy can titrate AR agonist DHT (0.5-10 nM) concentration ability to activate AR-transcription. LAPC4-GFP cells were infected with PSEBC-TSTA in media containing 0.5 to 10 nM of DHT. Seventy-two hours post-treatment, the cells were either lysed to be read by a conventional luminometer or imaged by bioluminescence microscopy (exposure time: 2 min). Sum grey intensity was normalized by number of fl-expressing cells (Sum grey intensity=sum grey intensity per ROI÷number of fl-positive cells). Firefly and GFP-expressing cells were counted using the cellSens software. Percentage of detected cells=(number of fl-positive cells÷number of GFP-expressing cells)×100. Relative fl activity (RLU) was normalized by protein content (RLU=RLU/μg protein). Data represent technical triplicates±SD.

FIG. 8 Imaging single cell heterogeneous responses to AR agonist and antagonists by using bioluminescence microscopy. (A) PSEBC-TSTA detected single cell heterogeneous responses to DHT and Enz in AR-responsive LAPC4 cells. LAPC4-GFP cells were infected with PSEBC-TSTA in media containing DHT (1 nM). Forty-eight hours post-infection, the cells were imaged after D-luciferin addition. After imaging, the media was changed and the treatments started (DHT (1 nM) or DHT+Enz (1 nM+10 μM)). GFP biofluorescence imaging was then performed every 5 h to track the cells. After 48 hours of treatment, luciferase imaging was repeated to determine the change in fl expression. Lower panels show representative single cell luminescence signals before and after treatments. The corresponding cells tracked and imaged by biofluorescence microscopy is also shown. (B) Sum of single cell LAPC4-GFP activity upon AR agonist (DHT) or antagonist (Enz or Bic) treatment. (C) PCA3 promoter activity is not modulated by antiandrogen treatment. LAPC4-GFP cells were infected with PCA3-3STA, treated and imaged as described in (A). (D) Upper panel: Scheme of the method used to isolate and image spiked PCa cells from blood. Spiked LAPC4-GFP cells were isolated from blood of a healthy donor and were infected with PSEBC-TSTA in media containing DHT (1 nM). Forty-eight hours post-infection, the cells were imaged and the media was changed to start the treatments (DHT (1 nM), DHT+Bic (1 nM+10 μM) or DHT+Enz (1 nM+10 μM)). Every 5 h, GFP biofluorescence imaging was performed to track the cells. At 48 h, luminescence imaging was repeated to determine single cell fl expression changes. Lower panels: Biofluorescence and bioluminescence images of blood spiked-LAPC4-GFP cells transduced with PSEBC-TSTA after PCa cell isolation. (E) Bioluminescence microscopy quantification of single cell responses to AR-antagonists after PSEBC-TSTA transduction of PCa spiked cells (LAPC4-GFP). Relative grey intensity=((sum grey intensity per ROI−sum grey intensity of background)÷sum grey intensity at 0 h)×100. Data represent technical triplicates±SD.

FIG. 9 Optimization of D-luciferin concentration for bioluminescence microscopy. (A) 22Rv1-GFP cells were infected with CMV-TSTA adenovirus. Seventy-two hours post-infection, bioluminescence microscopy imaging was performed after cell exposure to medium containing 0.88, 1.75, 3.5, 8.75 or 17.5 mM of D-luciferin. (B) Cell viability assays reveal that high D-luciferin concentrations are toxic for PCa cells. After imaging described in (a), viability assays were performed using Prestoblue reagent for each condition. (C) Biofluorescence (upper panel) and bioluminescence (lower panel) imaging (20×) of 22Rv1-GFP cells after CMV-TSTA transduction. Changes in cell morphology are seen at higher D-luciferin concentrations. (D) Sustained luciferase signal between 20 to 120 min after 3.5 mM D-luciferin exposure. 22Rv1-GFP cells were infected with CMV-TSTA adenovirus. Images were taken every 5 min for 160 min after D-luciferin (3.5 mM) containing medium exposure. Luminescent cells were counted by means of the cellSens software. Sum grey intensity was normalized by total number of fl-expressing cells (Sum grey intensity=sum grey intensity per ROI÷ number of fl-positive cells). Data represent technical triplicates±SD.

FIG. 10 Optimization of viral transduction conditions for PCa cell lines. (A-D) Percentage of detected cells for 22Rv1 (AR+), LNCaP (AR+), LAPC4 (AR+) and DU-145 (AR−) PCa cells at each time point. Firefly luciferase- and GFP-expressing cells were counted by means of the cellSens software. PCa cells (22Rv1, LNCaP, LAPC4 and DU-145) stably expressing GFP were transduced with increasing number of infectious viral particles of CMVTSTA, PSEBC-TSTA, and PSEBC-fl (10³-10⁷ ivp). Imaging was performed at 24, 48 and 72 h after an exposure time of 5 min per image using the bioluminescence microscope (LV200). Percentage of detected cells=(number of fl-positive cells÷number of GFP-expressing cells)×100. Data represent technical triplicates±SD.

FIG. 11 Optimization of exposure time to be used for bioluminescence imaging. 22Rv1-GFP cells were transduced with 10⁴ infectious viral particles of CMV-TSTA. Seventy-two hours after infection, imaging was performed at exposure times of 5, 10 and 20 min per image under bioluminescence microscopy. (A) The number of detected cells did not change with increasing exposure time. Graph shows no change in the percentage of detected cells for CMV-TSTA with increasing exposure time. (B) Graph shows the increase in the sum grey intensity of 22Rv1-GFP single cells with increasing exposure time. Data represent technical triplicates±SD.

FIG. 12 Growth curves of PCa cells treated with either bicalutamide (Bic) or enzalutamide (Enz). Twenty-four hours after seeding, LNCaP, LAPC4 and 22Rv1 cells were treated (DHT (1 nM), DHT+Bic (1 nM+10 μM) or DHT+Enz (1 nM+10 μM)). The cells were trypsinized and counted using a cell counter after six days of culture. Relative cell count=number of cells at each time point÷number of cells at 0 h. Data represent technical triplicates±SD. (A) LNCaP cells are growth inhibited by Enz. (B) LAPC4 cells are growth inhibited by both Bic and Enz. (C) 22Rv1 cells are not inhibited by Bic or Enz.

FIG. 13 Comparison of bioluminescence microscopy and conventional luciferase assays to measure AR-transcription activity changes associated with DHT or Enz treatments in PCa cells. AR-responsive LAPC4-GFP cells were infected with 10⁴ ivp of PSEBC-TSTA in media containing various treatments: Vehicle, DHT (1 nM) or DHT+Enz (1 nM+10 μM). Seventy-two hours after treatment, luciferase activity was measured by conventional luciferase assays (luminometer, (A) or by bioluminescence microscopy after D-luciferin exposure (LV200 microscope, (B). Relative fl activity (RLU) was normalized by protein content in each well (RLU=RLU/μg of protein). Sum grey intensity was normalized by total number of fl-expressing cells (Sum grey intensity=sum grey intensity per ROI÷number of fl-positive cells, (B). Each result represents technical triplicates±SD.

FIG. 14 Single cell bioluminescence microscopy using PSEBC-TSTA detected heterogeneous responses to treatment. AR-responsive PCa cells LAPC4, LNCaP and 22Rv1 stably expressing GFP were infected with 4 PSEBC-TSTA in media containing 1 nM of DHT. Forty-eight hours after infection, the cells were imaged by adding D-luciferin and an exposure time of 15 secs per image under bioluminescence microscopy. After imaging, treatments were started (vehicle, DHT (1 nM), DHT+Bic (1 nM+10 μM) or DHT+Enz (1 nM+10 μM)). GFP biofluorescence imaging was performed every 5 h to track the cells. After 48 hours, luminescence imaging was performed again on the same cells to determine the changes in fl activity. Relative grey intensity was quantified for n=10 cells in each treatment condition before and after treatment. Relative grey intensity=((sum grey intensity per ROI−sum grey intensity of background)÷sum grey intensity at 0 h)×100. Each ROI defines a single cell. Changes in fl activity in LAPC4 (A), LNCaP (B) and 22Rv1 (C) cells in response to treatment.

FIG. 15 LNCaP cells displaying greater cell-to-cell AR-transcriptional homogeneity upon treatment when compared to LAPC4 cells. Spiked LNCaP-GFP cells were isolated from blood of a healthy donor and were infected with PSEBC-TSTA in media containing 1 nM of DHT. Forty-eight hours after infection, the cells were imaged after an exposure time of 15 secs per image under bioluminescence microscopy. After imaging, the media was changed to start the treatments (DHT (1 nM), DHT+Bic (1 nM+10 μM) or DHT+Enz (1 nM+10 μM)). Biofluorescence microscopy imaging was performed for each well for 48 h, with images taken every 5 h to track the cells. Luciferase imaging was performed again on the same cells to determine the change in fl expression. Relative grey intensity=((sum grey intensity per ROI−sum grey intensity of background)÷sum grey intensity at 0 h)×100.

FIG. 16 PCA3-Cre-PSEBC-ITSTA could specifically detect and assess response to treatment of spiked PCa cells in urine of patients. (A) Scheme for targeting spiked PCa cells from urine. (B) PCa cells (22Rv1 stably expressing mCherry) were spiked in the urine of an healthy individual. Cells were recovered from the sample and transduced with CMV-TSTA or PCA3-Cre-PSEBC-ITSTA in media containing DHT (10 nM). Seventy-two hours after infection the cells were imaged by adding D-luciferin substrate and with an exposure time of 5 min per image using the bioluminescence microscope. (C) & (D) Graphs show single cell response to treatment detected with PCA3-Cre-PSEBC-ITSTA in LAPC4 and 22Rv1 cells. Urine samples from healthy patients were spiked with 250 PCa cells stably expressing GFP (22Rv1-GFP or LAPC4-GFP). Urine was spun down to collect all the cells. These cells were infected with 5×10⁵ virus particles of PCA3-Cre-PSEBC-ITSTA. Seventy-two hours after infection imaging was done using the bioluminescence microscope and post-imaging treatments started (DHT (1 nM), DHT (1 nM)+Bic (10 μM) or DHT (1 nM)+Enz (10 μM)). Forty-eight hours post treatment imaging was repeated to assess response on the same cells to determine change in luciferase expression. Relative grey intensity=((sum grey intensity per ROI−sum grey intensity of background)÷sum grey intensity at 0 h)×100). (E) Graph represents the sum of grey intensity of 10 cells after treatment (DHT (1 nM), DHT+Bic (1 nM+10 μM) or DHT+Enz (1 nM+10 μM)) for 22Rv1 and LAPC4 cells. (F) Graphs shows the relative percentage of LAPC4 or 22Rv1 cells without androgen receptor (AR) transcriptional activity inhibition by antiandrogen (bicalutamide or enzalutamide). Relative percentage=(percentage of cell with increasing luciferase activity after antiandrogen (bicalutamide or enzalutamide) treatment÷percentage of cell with increasing luciferase activity after androgen (DHT) treatment)×100.

FIG. 17 PCA3-Cre-PSEBC-ITSTA could specifically detect PCa cells in urine/bladder wash of patients. (A) Pathological and haematological traits of the patients tested with the number of detected cells. (B) Graphs shows the number of bioluminescence expressing cells in each patient group. Urine/bladder wash samples from PCa patients were collected. Cells were isolated from the samples and infected with PCA3-Cre-PSEBC-ITSTA. Imaging was done using the bioluminescence microscope with 3.5 mM D-luciferin and an exposure time of 20 seconds per field of view. Numbers of luminescent cells were counted using the CellSens software. (C) Image shows viable PCa cells in brightfield (left) and also expressing bioluminescence (right). (D) Immunofluorescence staining with multiple markers DAPI, Nucleolin, NKX3.1 and AMACR shows that the clump of cell expressing bioluminescence consists of PCa cells. Post-imaging the cells were fixed in paraformaldehyde. The cells were exposed to antibodies against Nucleolin, NKX3.1 and AMACR as well as staining for DAPI. The wells were then imaged using fluorescence microscope.

FIG. 18 PCA3-Cre-PSEBC-ITSTA could specifically detect and assess response to treatment of PCa cells in urine/bladder wash of patients. (A) & (B) Images show the H&E staining and IHC using anti-PSA for castration sensitive prostate cancer (CSPC) and castration resistance prostate cancer (CRPC) patients, case 9 and 11 respectively. (C), (D), (E) & (F) Graphs show single cell response to treatment detected with PCA3-Cre-PSEBC-ITSTA in PCa cells from case 9 and 11. Urine/bladder wash from patients post-DRE was sampled and spun down to collect all the cells. These cells were infected with 5×10⁵ virus particles of PCA3-Cre-PSEBC-ITSTA. Seventy-two hours after infection imaging was done with a bioluminescence microscope and post imaging treatment with AR agonist and antagonist was started (DHT (1 nM), DHT (1 nM)+Bic (10 μM) or DHT (1 nM)+Enz (10 μM)). Forty-eight hours post treatment imaging was repeated to assess response on the same cells to determine change in expression. Relative grey intensity=((sum grey intensity per ROI−sum grey intensity of background)÷ sum grey intensity at 0 h)×100). (G) Graphs shows the relative percentage of cells from case 9 or 11 without androgen receptor (AR) transcriptional activity inhibition by antiandrogen (bicalutamide or enzalutamide). Relative percentage=(percentage of cell with increasing luciferase activity after antiandrogen (bicalutamide or enzalutamide) treatment÷percentage of cell with increasing luciferase activity after androgen (DHT) treatment)×100.

FIG. 19 System to evaluate the dynamic response of single prostate cancer cells to antiandrogen. (1) Sampled urine or bladder wash are centrifuged and all the cells are collected. The cells are then infected with the MP-ITSTA system without additional enrichment or purification because of the high specificity of the system. (2) The first imaging is done at time 0 h (72 h after infection) to measure the basal luciferase activity reflecting the activity of androgen receptor. (3) Treatment initiation: DHT (control to determine non-specific cell death), DHT+bicalutamide, DHT+enzalutamide or any other drug that targets androgen receptor activity or PSA expression. (4) Second imaging is done at time 48 h post-treatment. (5) Single cell analysis: the level of luciferase expression at the second imaging is normalized with the first imaging for each cell. This step allows the dynamic evaluation of response to antiandrogen cell per cell. (6) Single cell analysis compilation to evaluate the potential response of the patient to the treatment.

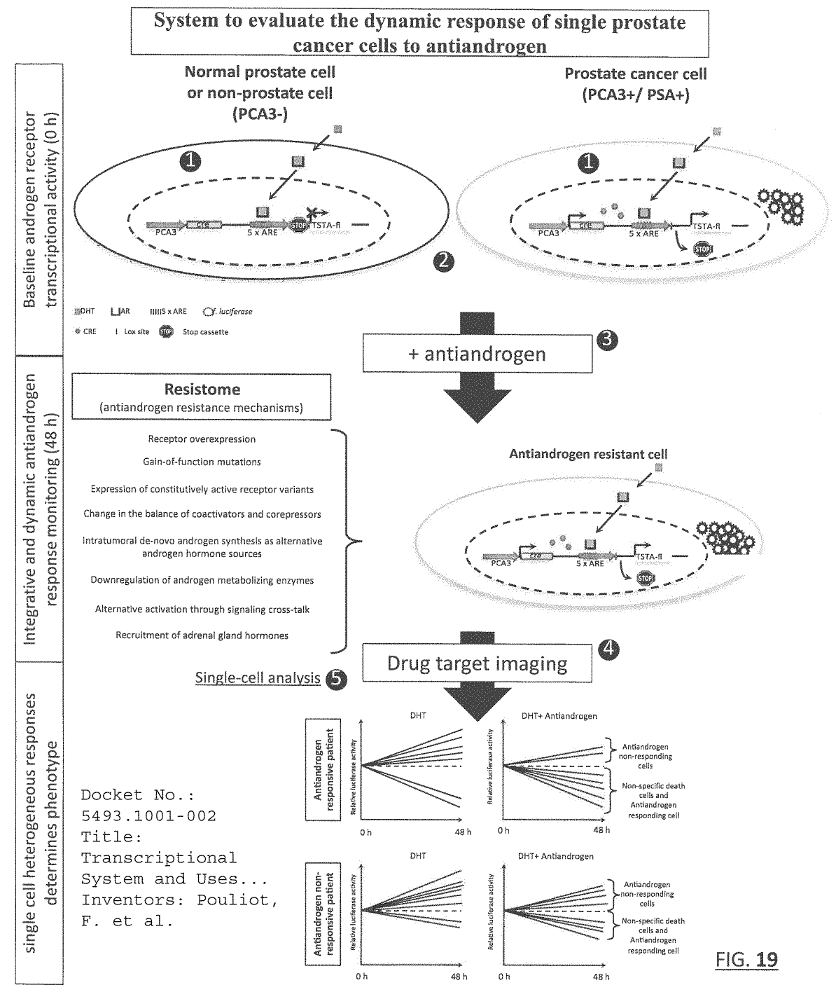

FIG. 20 Nucleolin, NKX3.1 and AMACR staining could distinguish between prostate cancer cell lines and bladder cancer cell lines. Immunofluorescence staining with multiple markers DAPI, Nucleolin, NKX3.1 and AMACR showed higher expression and were colocalized in prostate cancer cells. Prostate cancer cells (LAPC4, 22Rv1, LNCaP) and bladder cancer cells (T24, SW1710, SW780) were seeded in a 384 well plate. Twenty-four hours after seeding the cells were fixed in paraformaldehyde. The cells were exposed to antibodies against Nucleolin, NKX3.1 and AMACR as well as staining for DAPI. The wells were then imaged at 40× magnification using fluorescence microscope.

FIG. 21 Viability assay for PCa cells on treatment with either bicalutamide or enzalutamide. LAPC4 cells displayed inhibited growth with both Bic and Enz whereas 22Rv1 cells did not display inhibited growth with both Bic and Enz. LAPC4, and 22Rv1 cells were seeded. Twenty-four hours later, the cells were treated (DHT (1 nM), DHT+Bic (1 nM+10 μM) or DHT+Enz (1 nM+10 μM)). The cells proliferation was measured using resazurin. Relative cell count=(Fluorescence at each time point÷fluorescence at 0 h). Data represents technical triplicates±SD.

FIG. 22 Represents the scheme for isolation of prostate cancer cell from urine of patients and quantification of single cell treatment response of these detected cells (Matrigel™).

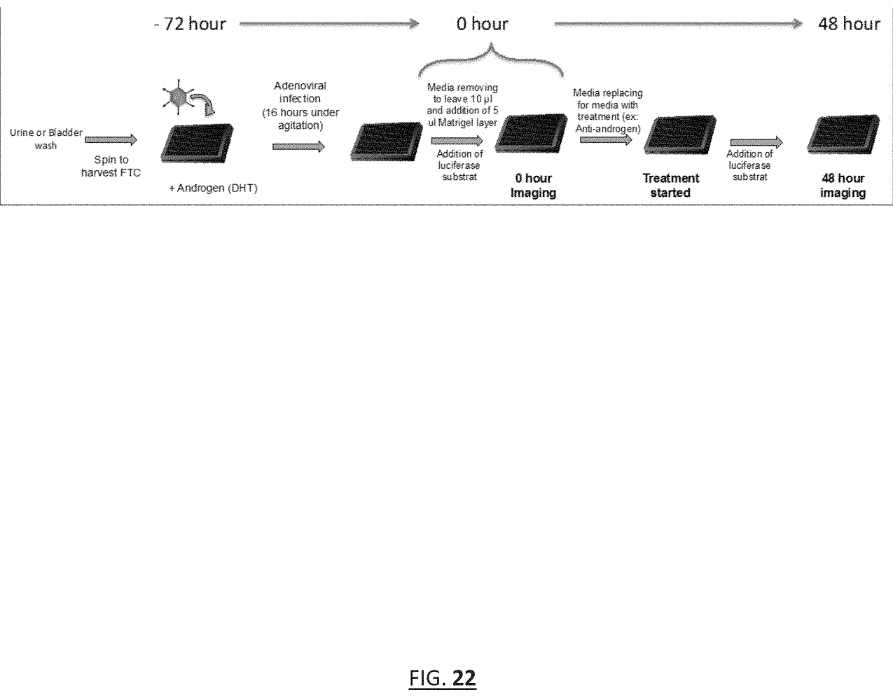

FIG. 23 The effect of the addition of a matrix, namely 10 μl Matrigel™, on the immobilization of the cells observed by bioluminescence. (A) The addition of Matrigel™ (20% and 30% final) results in a decrease of around 30% of the disturbed cells count after media addition. Displaced and lost cells are considered disturbed cells. (B) The relative number of disturbed cells is also reduced after media addition in the presence of Matrigel™. (C) The addition of Matrigel™ (20% and 30% final) results in an increase of around 30% of the identified cells count 48 h after media addition. (D) The relative number of identified cells is also increased in the presence of Matrigel™.

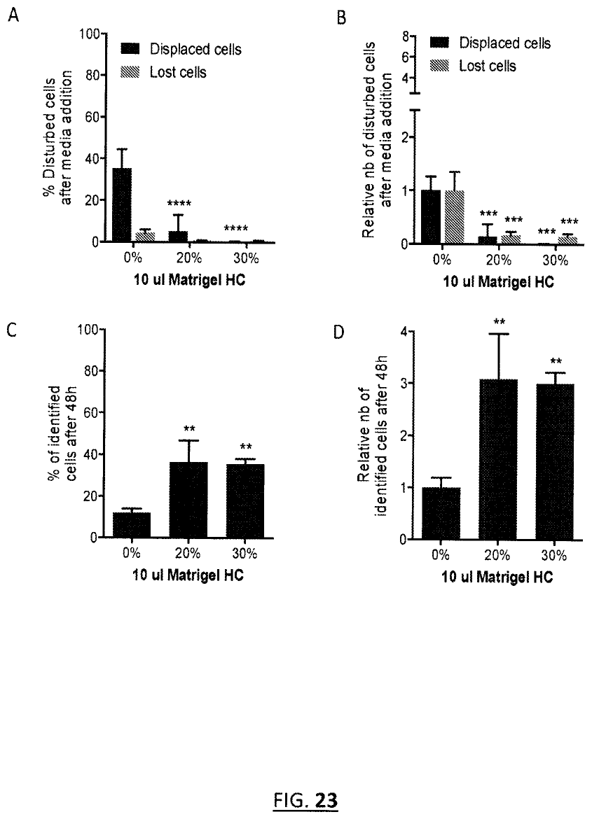

FIG. 24 PCA3-Cre-PSEBC-ITSTA could specifically detect prostate cancer (PCa) cells from urine/bladder wash/body fluid of patients and assess their resistance to treatment (A) or highlight potential agonist effect of antiandrogen. (A) Serum PSA levels of patient 22 during its treatment with bicalutamide or enzalutamide. (B) Relative fraction of androgen receptor (AR) and (C) Relative mean luminescence activity per cell show a greater resistance profile to bicalutamide than enzalutamide. (D) Images show the H&E staining and IHC using anti-PSA for castration resistance prostate cancer (CRPC) patients 11. (E) Relative fraction of androgen receptor (AR) and (F) Relative mean luminescence activity per cell indicate a potential agonist effect of bicalutamide on prostate cancer cells from case 11. Urine or bladder wash from patients was sampled and spun down to collect all the cells. These cells were infected with 5×105 virus particles of PCA3-Cre-PSEBC-ITSTA. Seventy-two hours after infection imaging was done with a bioluminescence microscope and post imaging treatment with AR agonist and antagonist was started (DHT (1 nM), DHT (1 nM)+Bic (10 μM) or DHT (1 nM)+Enz (10 μM)). Forty-eight hours post treatment imaging was repeated to assess response on the same cells. Relative fraction of AR active cells=(percentage of cell with luciferase activity after antiandrogen (bicalutamide or enzalutamide) treatment÷percentage of cell with luciferase activity after androgen (DHT) treatment)×100. Relative mean luminescence activity per cell=(percentage of mean luminescence activity per cell after antiandrogen (DHT, bicalutamide or enzalutamide) treatment÷percentage of mean luminescence activity per cell after DHT treatment)×100.

FIG. 25 The continuous monitoring of cells activity following treatment (DHT).

### DETAILED DESCRIPTION

### Sequence Listing

TABLE 1

| SEQ ID NO: | Name |
|---|---|
| 1 | PCA3 Promoter |
| 2 | PSEBC Promoter |
| 3 | STOP Sequence |
| 4 | CRE Recombinase |
| 5 | BGH-Ig Intron |
| 6 | PCA3-CRE-PSEBC-ITSTA |
| 7 | PEG3AP1 Promoter |
| 8 | PEG3wt Promoter |
| 9 | CRE Forward Primer (1-465) |
| 10 | CRE Reverse Primer (1-465) |
| 11 | CRE Forward Primer (466-1523) |
| 12 | CRE Reverse Primer (466-1523) |
| 13 | Human Chimeric Intron Forward Primer |
| 14 | Human Chimeric Intron Reverse Primer |
| 15 | Mice Protamine Intron Forward Primer |
| 16 | Mice Protamine Intron Reverse Primer |
| 17 | Mice Protamine Intron AG Forward Primer |
| 18 | Mice Protamine Intron AG Reverse Primer |
| 19 | PSEBC Forward Primer |
| 20 | PSEBC Reverse Primer |
| 21 | Stop Cassette Forward Primer |
| 22 | Stop Cassette Reverse Primer |
| 23 | Stop Cassette Probe |
| 24 | Fl Gene Forward Primer |
| 25 | Fl Gene Reverse Primer |
| 26 | Fl Gene Probe |

### Definitions

The expression “dynamic assessment” refers to the monitoring of cells (e.g. behavior, viability, proliferation, cytotoxicity, drug response etc.) over time. Dynamic assessment includes the monitoring (continuous or at specific time points) of the behavior, viability, proliferation, cytotoxicity, drug response of cells over a given period of time.

The term “biological fluid” refers to liquids originating from the human body. Biological fluids include blood, urine, peritoneal fluid, pleural fluid, cerebrospinal fluid, amniotic fluid, gastric juice, mucus, pericardial fluid, saliva, semen and vaginal secretion. Preferably, the fluid is blood or urine.

The term “construct” refers to a molecule composed of nucleotide monomers covalently bonded in a chain. Said construct can be engineered to contain nucleotide sequences of interest. Methods for preparing polynucleotide constructs are well known in the art such as using common enzymes for instance restriction endonuclease and ligase. In one aspect, the construct is a virus or a plasmid. In a further aspect, the construct is an adenovirus or a replication-defective virus.

The “first” and “second” systems/promoters are independently chosen from tissue specific, cancer specific and treatment response imaging systems or promoters. The first and second systems/promoters may be chosen to allow to genotype specificity.

The term “tissue specific promoter” refers to a promoter whose transcriptional activity is specific to cells or tissues. Tissue specific promoter include PCA3 promoter.

The term “cancer specific promoter” refers to a promoter whose transcriptional activity is specific to cancer cells or tissues. Cancer specific promoters include PCA3, PEG3, PEG3AP1, MUC1, N-Myc or BRN2.

The term “treatment response imaging system” refers to a system whose transcriptional activity is modulated by a given treatment or drug target, either, directly, or through a genetically engineered system involving for example phosphorylation and/or protein/protein interaction. In one aspect, the treatment response imaging system is a response treatment promoter. In one aspect, response treatment promoters include PSA and PSEBC promoters.

In one aspect, the treatment is an anti-cancer treatment. In a further aspect, the treatment is androgen deprivation therapy (ADT).

The term “inserting” refers to the delivery of a construct from a vector to a cell either by transfection or transduction.

The term “PCA3 promoter” refers to a DNA sequence that initiates transcription of PCA3 gene (Prostate Cancer associated 3; see for example Accession Nos: NR_132312.1, NR_132313.1 and NR_015342.2). The sequence the PCA3 promoter and variants thereof are known; see for example Genebank Accession Nos: KT596747.1, KT596746.1, KT596745.1, KT596744.1 and KT596743.1, and Neveu, B. et al. 2016. In some embodiments, the sequence of the PCA3 promoter may be at least 70%, 75%, 80%, 85%, 90%, or 95% identical to sequence of SEQ ID NO: 1. In some embodiments, the sequence of the PCA3 promoter comprises a nucleic acid at least 70%, 75%, 80%, 85%, 90%, or 95% identical to sequence of SEQ ID NO: 1. In one aspect, the sequence of the PCA3 promoter is depicted in SEQ ID. NO: 1.

The term “PSA promoter” refers to a DNA sequence that initiates transcription of PSA gene (Prostate Specific Antigen GenBank: X14810.1). In a preferred embodiment, the PSA promoter is a chimeric promoter (PSEBC) which comprises the ARE-bearing promoter see for example Wu et al. 2001 and Pouliot et al. 2009. The sequence and characterization of the PSA and PSEBC promoter are known see for example Genebank Accession Nos AF394907. In some embodiments, the sequence of the PSA promoter may be at least 70%, 75%, 80%, 85%, 90%, or 95% identical to sequence of SEQ ID NO: 2. In some embodiments, the sequence of the PSA promoter comprises a nucleic acid at least 70%, 75%, 80%, 85%, 90%, or 95% identical to sequence of SEQ ID NO: 2. In one aspect, the sequence of the PSA promoter is depicted in SEQ ID. NO: 2.

“Site-specific recombinase” refers to a recombinase which recognize cognate site-specific recombination sequence and operates in mammalian cells. Recombinases include Cre recombinase (locus loxP and loxM, element), Flp recombinase (short flippase recognition (FRT) target) and other recombinases such as øC31 and cas9 from the CRISPR/Cas9 system. In one embodiment, an intron such as a BGH intron (See SEQ ID. NO: 5) inserted in the sequence of the site-specific recombinase to avoid leaky expression of wild-type site-specific recombinase causing non-specific cleavage of the cognate site-specific recombination sequence in prokaryotic bacterial system.

In a preferred embodiment, the site-specific recombinase is Cre which is a 38 kDa Type I topoisomerase protein from bacteriophage P1 (see for example GenBank sequence YP 006472) which mediates intramolecular recombination. The enzyme recognises the cognate site-specific recombination sequence loxP (locus of crossing over of P1) and causes recombination between these pairs of sites. In some embodiments, the sequence of the site-specific recombinase may be at least 70%, 75%, 80%, 85%, 90%, or 95% identical to sequence of SEQ ID NO: 4. In some embodiments, the sequence of the site-specific recombinase comprises a nucleic acid at least 70%, 75%, 80%, 85%, 90%, or 95% identical to sequence of SEQ ID NO: 4. In one aspect, the sequence of the site-specific recombinase is depicted in SEQ ID NO: 4.

“Cognate site-specific recombination sequence” refers to a DNA sequence recognized by a site-specific recombinase. In one embodiment, the cognate site-specific recombination sequence a loxP-STOP-loxp sequence. In a further aspect, the STOP sequence may be at least 70%, 75%, 80%, 85%, 90%, or 95% identical to sequence of SEQ ID NO: 3. In some embodiments, the STOP sequence comprises a nucleic acid at least 70%, 75%, 80%, 85%, 90%, or 95% identical to sequence of SEQ ID NO: 3. In one aspect, the STOP sequence is depicted in SEQ ID NO: 3.

The expression “under the control of a promoter” refers to the promoter capable of causing expression of a sequence downstream to it such as a reporter or a site-specific recombinase. Techniques for operatively linking different components in a polynucleotide construct are well known to those skilled in the art. Such techniques may include the use of linkers such as synthetic linkers, for example including one or more restriction enzyme sites.

The term “amplification system” refers to a system comprising at least one activator sequence and at least one responsive promoter sequence.

The term “activator sequence” refers to a nucleotide sequence encoding a polypeptide which activates the responsive promoter sequence. In one embodiment, the activator sequence can comprise a sequence encoding GAL4 DNA binding domain and VP16 transactivation domain.

The expression “responsive promoter sequence” refers to a promoter that can be bound and activated by the activator. In one embodiment, the responsive promoter sequence is at least one GAL4 and preferably 5 GAL4.

The expression “bioluminescent reporter gene” refers to a gene that encoding an enzyme capable of catalyzing photon-emitting substrates. Preferably, the enzyme is a luciferase, for example Firefly luciferase, Beetle luciferase, Renilla luciferase, Gaussia luciferase, Metridia luciferase, Vargula luciferase, bacterial luciferase, or Nano-luciferase 1.1. In one aspect, the bioluminescent substrate is a photon-emitting substrate chosen from D-luciferin, Coelentrazine, Vargula luciferin, a fatty acid or Furimazine.

The expression “upstream” refers to a sequence localised 5′ to the specified sequence whereas the expression “downstream” refers to a sequence localised 3′ to the specified sequence.

The expression “replication-defective virus” refers to a virus which cannot completely replicate. Such virus can infect cells but cannot replicate upon infection of said cells. Said replication-defective virus includes for instance, mutant variants of adenovirus, adeno-associated virus, herpes virus, vaccinia virus, polio virus, AIDS virus, neuronal trophic virus and sindbis retrovirus such as Murine Maloney Leukemia Virus. Methods for producing such replication-defective virus are known to a person skilled in the art.

In one aspect, the present description relates to a PCA3-PSA construct for transducing prostate cancer cells comprising:
a Cre recombinase under the control of a PCA3 promoter;
a bioluminescent reporter gene under the control of a PSA promoter; and
a Cre site-specific recombination sequence, said Cre site-specific recombination sequence located upstream of the bioluminescent reporter gene;
wherein, upon activation of the PCA3 and PSA promoters, said transduced cancer cells expresses the bioluminescent reporter gene following recombination.

In some embodiments, the sequence of the PCA3-PSA construct may be at least 70%, 75%, 80%, 85%, 90%, or 95% identical to sequence of SEQ ID NO: 6. In some embodiments, the sequence of the PCA3-PSA construct comprises a nucleic acid at least 70%, 75%, 80%, 85%, 90%, or 95% identical to sequence of SEQ ID NO: 6. In one aspect, the sequence of the PCA3-PSA construct is depicted in SEQ ID NO: 6.

The term “cancer” includes:

TABLE 2
acute lymphocytic cancer (ACL);
anal cancer;
basal cell carcinoma;
bladder cancer;
brain tumors including gliomas;
breast cancer;
cervical cancer;
Choriocarcinoma;
chronic myeloid chronic myeloid leukemia (CML);
colon cancer;
colorectal cancer;
diffuse large B-cell lymphoma;
endometrial cancer esophageal cancer;
esophageal cancer;
gallbladder cancer;
gastric cancer;
gastrointestinal cancers including gastrointestinal stromal tumors;
head and neck cancer;
liver cancer;
lung cancer including small cell lung cancer and non-small cell lung
cancer;
lymphocytic lymphomas;
lymphomas including follicular lymphoma;
lymphomas including Hodgkin's disease;
multiple myeloma;
myelogenous leukemia including acute myeloid leukemia (AML);
myeloproliferative disorders;
neuroblastomas follicular lymphoma;
oral cancer;
ovarian cancer;
pancreatic cancer;
prostate cancer;
rectal cancer;
renal cancer;
Sarcomas;
skin cancers such as melanoma;
stomach cancer;
T-cell leukemia lymphoma;
testicular cancer; and
thyroid cancer.

The expression “prostate cancer” refers to a form of cancer developing in the prostate. There are few types of prostate cancers such as adenocarcinoma and neuroendocrine carcinoma. Some prostate cancers can be androgen-independent or androgen-dependent.

The following embodiments are present alone or in combination:

In one aspect, the methods and uses of the description are non-invasive.

In one aspect, the sequence of the PCA3 promoter comprises a nucleic acid at least 70%, 75%, 80%, 85%, 90%, or 95% A identical to sequence of SEQ ID NO: 1.

In one aspect, the sequence of the PSA promoter comprises a nucleic acid at least 70%, 75%, 80%, 85%, 90%, or 95% A identical to sequence of SEQ ID NO: 2.

In one aspect, the sequence of the Cre recombinase comprises a nucleic acid at least 70%, 75%, 80%, 85%, 90%, or 95% identical to sequence of SEQ ID NO: 4.

In one aspect, the Cre site-specific recombination sequence is a loxp site comprising a STOP sequence and the STOP sequence comprises a nucleic acid at least 70%, 75%, 80%, 85%, 90%, or 95% identical to sequence of SEQ ID NO: 3.

In one aspect, the sequence of the construct comprises a nucleic acid at least 70%, 75%, 80%, 85%, 90%, or 95% identical to sequence of SEQ ID NO: 6.

In one aspect, the construct comprises an amplification system comprising at least one responsive element sequence and an activator sequence said amplification system located upstream of the bioluminescent reporter gene.

In one aspect, the construct comprises an amplification system comprising at least one responsive element sequence and an activator sequence said amplification system located upstream of the bioluminescent reporter gene and downstream of the second or PSEBC promoter.

In one aspect, in the activator sequence encodes GAL4-VP16 or GAL4-VP2 polypeptide and wherein the responsive element sequence comprises GAL4RE sequence.

In one aspect, the bioluminescent reporter gene is luciferase. In one aspect, the bioluminescent substrate is luciferin.

In one embodiment, the harvested cancer cells may be immobilized (e.g. on an appropriate support). Techniques for immobilizing cells include adsorption, covalent binding, entrapment, co-polymerization and encapsulation. In one embodiment, the cells are immobilized with a matrix such as a reconstituted basement membrane preparation that is extracted from the Engelbreth-Holm-Swarm (EHS) mouse sarcoma (Matrigel™ from Corning).

In one aspect, the harvested cancer cells are immobilised with a matrix. In one aspect, the matrix is an extracellular matrix comprising a solubilized basement membrane preparation extracted from the Engelbreth-Holm-Swarm (EHS) mouse sarcoma (e.g. Matrigel™).

In one aspect, the cancer specific promoters are PCA3, PEG3, PEG3AP1, N-Myc or BRN2.

In one aspect, the response treatment system is a response treatment promoter (e.g. PSA or PSEBC promoter).

In one aspect, the site-specific recombinase is a recombinase which recognizes cognate site-specific recombination sequence and operates in mammalian cells.

In one aspect, the site-specific recombinase is Cre recombinase (locus loxP and loxM, element), Flp recombinase (short flippase recognition (FRT) target), øC31 or cas9 CRISPR/Cas9 system.

In one aspect, the site-specific recombinase is Cre recombinase comprising an intron such as a BGH intron (e.g. SEQ ID. NO:5).

In one aspect, the bioluminescent substrate is added once. In one aspect, the bioluminescent substrate is added to the culture medium before and after the treatment step.

In one aspect, the baseline reading and the post treatment reading are conducted on the same region of interest.

In one aspect, the baseline reading and the post treatment reading are conducted on the same single cancer cell.

In one aspect, in the methods of the description further comprises the step inserting (e.g. transducing) a construct as described herein in the harvested cancer cells of the patient shortly after collection of the biological fluid.

In one embodiment, the methods of the present description may be used to detect primary cancer cells. The primary cancer cells may be one of the cancer cells described herein.

When detecting/assessing prostate cancer cells from a urine sample, a Digital Rectal Examination (DRE) may be performed prior to the collection of the sample to increase shedding of prostate cells into the urine. Alternatively, the urine sample may be collected without a DRE.

In one embodiment, the present description relates to a method for identifying one or more single prostate cancer cells from cells harvested from the urine or blood of a patient, said method comprising:
providing a culture medium one or more harvested cancer cells transduced with a construct comprising:
   a Cre recombinase under the control of a cancer-specific promoter (e.g. PCA3 promoter);
   a bioluminescent reporter gene under the control of a system imaging the activity of a drug target (e.g. PSA promoter); and
   a Cre site-specific recombination sequence, said Cre site-specific recombination sequence located upstream of the bioluminescent reporter gene;
   wherein, upon activation of the cancer-specific promoter and drug target imaging system, said transduced cancer cells express the bioluminescent reporter gene following recombination;
adding a bioluminescent substrate and;
imaging harvested prostate cancer cells by bioluminescence microscopy to identify one or more single prostate cancer cells.

In one embodiment, the present description relates to a method for dynamically assessing the treatment response (e.g. antiandrogen response) of one or more single prostate cancer cells from cells harvested from the urine or blood of a patient, said method comprising:
providing a culture medium comprising one or more harvested prostate cancer cells transduced with a construct comprising:
   a Cre recombinase under the control of a cancer-specific promoter (e.g. PCA3 promoter);
   a bioluminescent reporter gene under the control of a system imaging the activity of a drug target (e.g. PSA promoter); and
   a Cre site-specific recombination sequence, said Cre site-specific recombination sequence located upstream of the bioluminescent reporter gene;
   wherein, upon activation of the cancer-specific promoter and drug target imaging system, said transduced cancer cells express the bioluminescent reporter gene following recombination;
adding a bioluminescent substrate;
imaging harvested prostate cancer cells by bioluminescence microscopy to get a baseline reading;
adding a bioluminescent substrate; and
treating harvested prostate cancer cells with a treatment (e.g. an antiandrogen treatment);
imaging harvested prostate cancer cells by bioluminescence microscopy to get a post-treatment reading; and
determining whether the one or more assessed cancer cells have responded to the treatment (e.g. antiandrogen treatment) based on the difference between the baseline reading and the post-treatment readings (e.g. interpreted as a unique data point or as a continuous variable).

In the present description, the present inventors have developed and characterised an imaging method to detect primary PCa cell from urine in order to determine single cell dynamic response to antiandrogens. This method combines bioluminescence microscopy and a transcriptional amplification system, which integrate the activities of the PCA3 and PSEBC gene promoters as a single output driving the firefly luciferase gene reporter. In this imaging system, the PCA3 promoter provides the specificity to PCa cells, while the PSEBC promoter could titer AR transcriptional activity. This method may be used to image heterogeneous antiandrogen cell responses both in castration-sensitive and castration-resistant cell lines or patients.

In one aspect, the cell imaging technique presented herein and more specifically in the Example section is distinct as it can monitor living single cell responses to antiandrogen therapy and does not require enrichment or selection of cells for isolation. In one aspect, the ability to image PCa cells despite heterogeneous cell population found in urine relies on the combined specificities of the promoters selected, a transcriptional amplification system and the unique accuracy of bioluminescence microscopy with minimal background and autofluorescence. In one aspect, most PCa cell genotypes shredding into urine could be imaged, as long as a single or a combination of promoters confer cell genotype specificity. Like any method relying on specific gene expression, this method relies on specific promoter activation which imply that some cancer cells will not be detected. For instance, in some embodiments, AR negative PCa cells may not be imaged with a PSA based system, but by selecting a luciferase reporter emitting photons at non-overlapping wavelengths, co-infection of urine or blood cells with transcriptional amplification systems imaging both AR negative and positive cells could overcome this limitation. Indeed, co-infection of urine cells with PCA3-3STA and PCA3-Cre-PSEBC-ITSTA systems expressing CBG and CBR luciferases, respectively, could identify both AR positive and negative adenocarcinomas. Moreover, neuroendocrine specific imaging systems based on N-Myc or BRN2 transcription factors co-infected with PCA3-3STA system could identify both adenocarcinoma and neuroendocrine cells in advanced PCa. Of note, while urine may be preferred, other body fluid could be used to target circulating tumour cells (CTCs). CTCs targeted using MP-ITSTA could give an account of CTC concentration and dynamic response to antiandrogens or other therapies. This may provide a better characterisation of the response to antiandrogens in metastatic PCa patients, given the new literature on intrapatient polyclonality (Beltran H et al. 2016, Gundem et al. 2015).

In one embodiment, the method presented herein, which is based on drug-target single cell imaging, has a unique ability to be highly integrative at molecular, cellular and cell population levels. It can detect molecular changes by monitoring the activity of AR in real-time. Through dynamic imaging of single cells AR activity upon antiandrogen exposure, this method can also integrate most antiandrogen resistance mechanisms (resistome) and their interactions together to escape from antiandrogen inhibitory effects. At cellular levels, our method could be used to image heterogeneous responses of AR positive cells to antiandrogens in order to determine patient's sensitivity to antiandrogens such as Enz. By including AR− cell imaging in the model, a number of static and dynamic cellular data upon antiandrogen exposure could be registered and serve as mathematical model to predict response to antiandrogen treatment.

Also, dynamic single cells studies using PCA3-Cre-ITSTA bioluminescence microscopy can be linked to omic studies and can help to understand better the genomic, transcriptomic and proteomic parameters involved in the antiandrogen resistance. These cells being shed directly from the tumor mirror the genotypic characteristics of the tumor. As opposed to cell culture, shorter time duration of culture can help to avoid differentiation and selection associated with ex-vivo environment.

When different cohorts of patients, primary PCa to CRPC were compared it was seen that the system could discriminate the overall response of a patient to antiandrogens. Further the single cell imaging enabled us to identify the outlier non-responsive population cells within each patient. It was seen that even though the whole cell population in primary PCa patient showed a decrease with AR antagonist (bicalutamide) treatment 50% of the cells showed an increase in fl expression. These non-responding populations of cells could act as an important target to study failure in drug response. Similarly, in the case of CRPC patient, 80% of the cells showed a decrease in expression with antiandrogen and only 20% cells were identified to be resistant suggesting that ADT treatment could still be given to this patient in combination with other therapies to improve the overall survival of the patient. Further when the cumulative mean grey intensity in presence of antiandrogens was compared among the two cohorts it could predict the overall response of the patients based on clinical characteristics.

In one embodiment, the system is specific for AR responsive prostate cancer cells and does not incorporate a population of cells that is AR non-responsive. In one aspect, the prostate cancer patient should not be a patient who has developed metastasis after Radical Prostatectomy (RP). In a further aspect, the patient is a prostate cancer patient who did not undergo prostatectomy before developing metastasis. However, analysis of CTC from blood could act as a surrogate of urine in these cases.

In one aspect, the present description shows that a system (e.g. MP-ITSTA) based on combinational activation of two specific promoters has the ability to detect specifically prostate cancer cells and study dynamic, integrative, and quantitative drug response in urine of PCa patients. The system can act as an important tool in providing personalised medicine to patients based on the single cell study of these disseminated cells.

Furthermore, the system is not limited to PCa cells but also can be expanded to other cancer types (See Table 2) by using tissue specific promoters along with regulatory elements for drug targets for each cancer type. In one embodiment, the cancer is bladder cancer or breast cancer.

The present invention will be more readily understood by referring to the following examples. These examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the preferred methods and materials are described. The issued patents, published patent applications, and references that are cited herein are hereby incorporated by reference to the same extent as if each was specifically and individually indicated to be incorporated by reference. In the case of inconsistencies, the present disclosure will prevail.

### EXAMPLES

### Example 1—Multi-Promoter Integrated Two-Step Transcriptional Amplification System (STA)

In order to generate a bioluminescence microscopy dynamic imaging nanosystem that is highly specific to prostate cancer (PCa) and that can give account of response to androgen deprivation therapies in viable primary PCa single cells harvested from urine, the transcriptional specificities of the PSEBC (androgen sensitive) and PCA3 (PCa specific) promoters were combined.

PCA3-Cre-PSEBC-ITSTA System

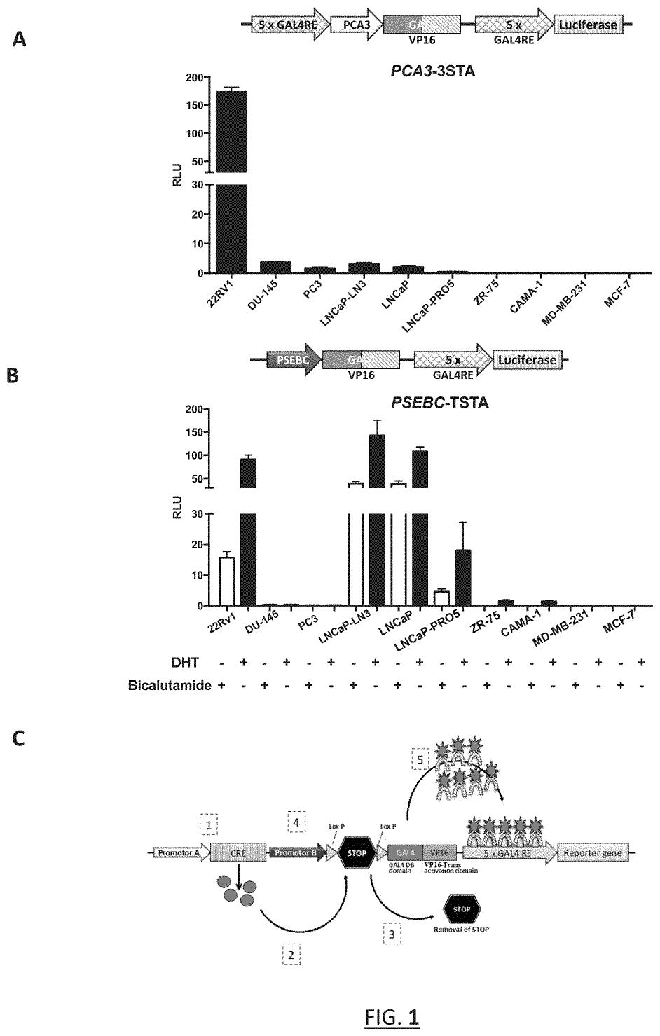

To exploit the combined potential of PCA3 and PSEBC promoter as a single output, the multi-promoter integrated two-step transcriptional amplification system (MP-ITSTA) was created (FIG. 1 C). MP-ITSTA utilizes the site-specific recombination ability of Cre recombinase to cleave the DNA fragment specifically between two loxP sites. The system consists of 4 major steps: 1) the activation of the first promoter leads to the production of Cre recombinase; 2) the Cre recombinase then identifies the loxP sites and cleaves the DNA fragment between them (which contains a Stop cassette); 3) a second promoter is activated and the GAL4VP16 protein is produced; 4) the GAL4VP16 fusion protein binds to GAL4RE upstream of the reporter gene and amplify promoter driven expression. Therefore, this system is designed to combine the specificity of two promoters to drive the expression of a single reporter gene after Two-step transcriptional amplification system (TSTA) transcriptional amplification (FIG. 1 C).

Identification of an Appropriate Stop Cassette

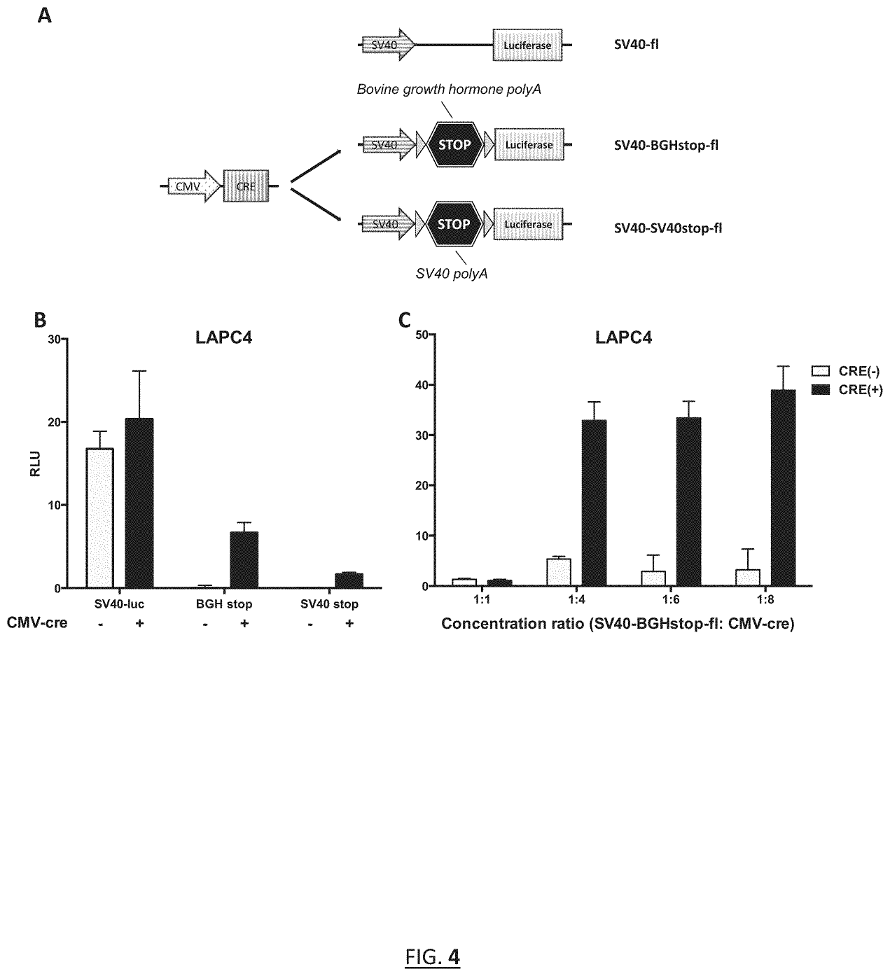

As a first step for the development of the MP-ITSTA system, an appropriate stop cassette had to be identified. A stop cassette is a sequence inserted between the loxP sites that would completely inhibit the luciferase expression in the absence of Cre recombinase. To this end, the bovine growth hormone (BGH) or Simian Virus 40 (SV40) polyadenylation (polyA) sequences were inserted between the loxP sites as stop cassettes and their ability to inhibit the ubiquitous SV40 promoter (FIG. 4) were compared. As shown, both sequences could block the SV40 promoter driven firefly luciferase (fl) gene in the absence of Cre recombinase. On co-transfection with plasmid expressing Cre recombinase, the BGH polyA sequence lead to a better reactivation of the system giving 4.04-times higher fl signal compared to the SV40 polyA sequence. Therefore, the BGH polyA sequence was used for the next experiments.

Generation of a Modified Cre Recombinase with the Insertion of an Intron

After identification of the appropriate stop cassette, an adenoviral backbone containing both the Cre recombinase and the two loxP sites in the same plasmid was generated. In concordance with earlier studies (Kaczmarczyk, 2001), it was not possible to amplify adenoviral backbone plasmids containing both the wild-type Cre recombinase and the loxP sites. This was secondary to leaky expression of wild-type Cre recombinase causing non-specific cleavage of loxP sites in prokaryotic bacterial system as previously described (Kaczmarczyk, 2001). This led to a loss of bacterial colonies having the intact viral backbones with all the desired coding sequences.

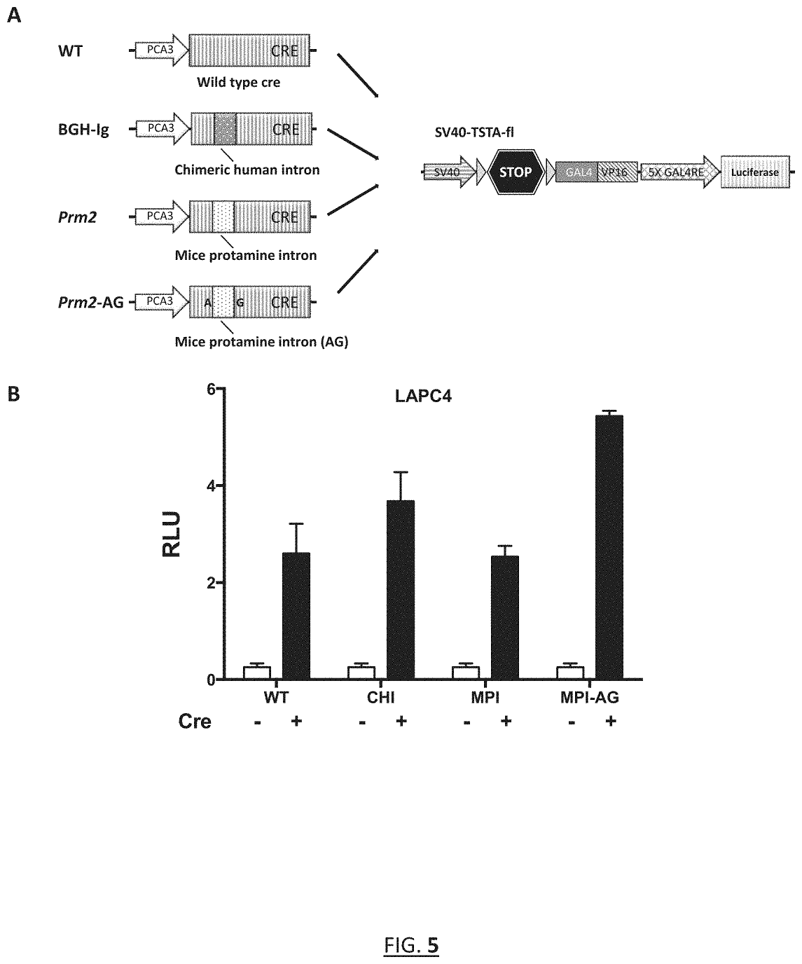

To inhibit bacterial expression of Cre recombinase, an intron was inserted 465 bp downstream of the Cre recombinase start site. The absence of post-transcriptional splicing machinery in E. Coli would prevent the expression of functional Cre recombinase and allow adenoviral DNA amplification. Three intron sequences inserted in the Cre recombinase cDNA were tested: 1) a human BGH-Ig chimeric intron (5′-donor site from the first intron of the human β-globin and the branch and 3′-acceptor site from the intron located between the leader and body of an immunoglobulin gene heavy chain variable region) (Kaczmarczyk, 2001); 2) the Prm2 (mice protamine 2 gene) intron (Kwon, 1993); and 3) the modified Prm2 containing eukaryotic splice site (AG). As shown in (FIG. 5 A), all three intron sequences, when inserted in the Cre recombinase cDNA, could enhance the expression after plasmid transfection. However, only the Cre recombinase containing chimeric human intron allowed amplification of adenoviral backbones in bacteria (FIG. 5 B). Therefore, the BGH-Ig chimeric human intron was used in all of the MP-ITSTA constructs created.

Relative Orientation of Each Component of the System

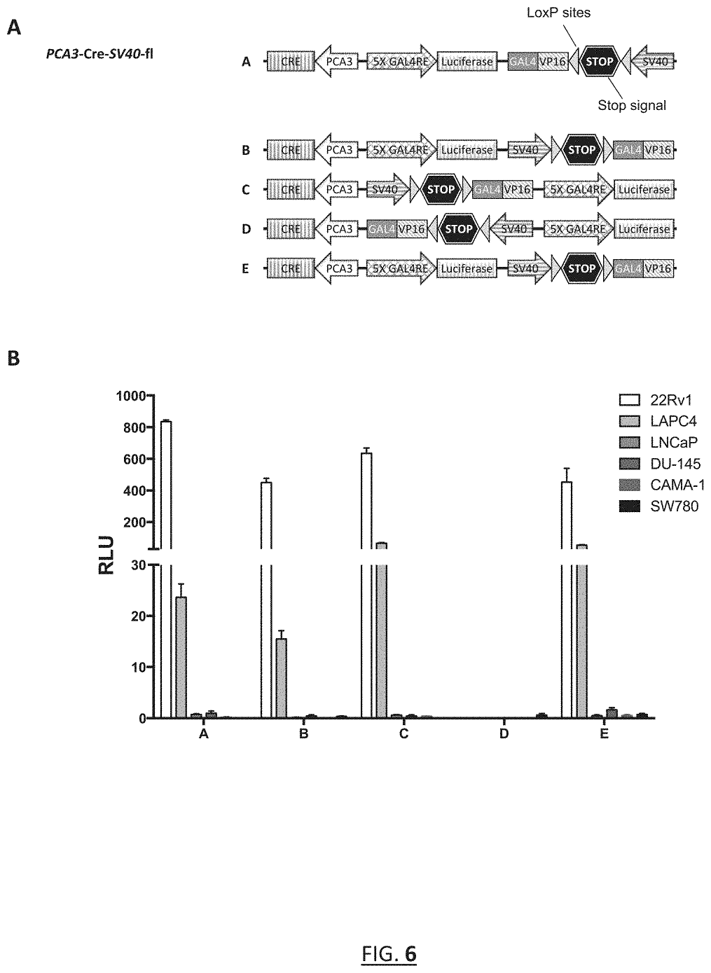

As a final step to construct a bi-promoter integrative imaging system, the optimum relative orientation of each component of the MP-ITSTA had to be determined. Fl activities obtained from systems where the activator and amplifier cassette were in several orientations were compared. For these experiments, the SV40 promoter was driving the TSTA and the PCA3 promoter was driving the Cre recombinase. After testing the system in 22Rv1, LAPC4, LNCaP, DU-145 (all PCa), CAMA-1 (breast cancer) and SW-780 (bladder cancer) cell lines, it was seen that orientation A provided the highest reporter gene signal, while being specific to PCa cells due to PCA3 driven Cre recombinase expression (FIG. 6).

Specificity of the System

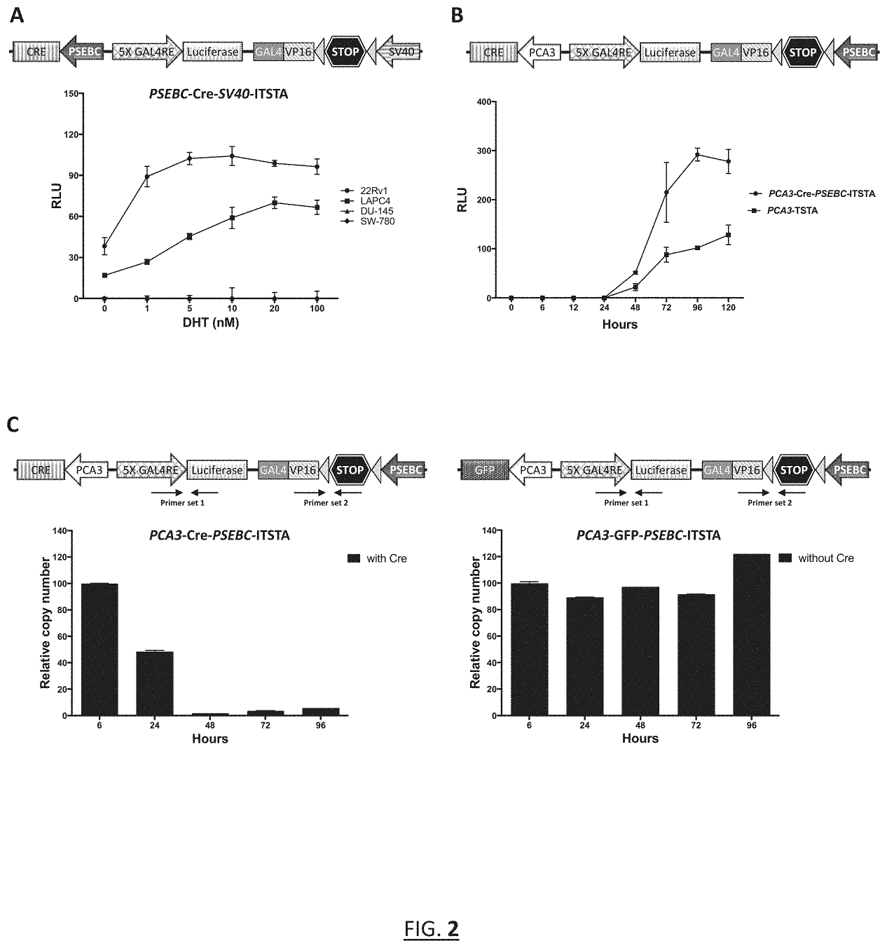

To characterize if the activity of the MP-ITSTA system was dependent on the activity of only the TSTA driving promoter or of the two promoters, a test was conducted to assess the ability of dihydrotestosterone (DHT) to activate PSEBC driving either the Cre recombinase (FIG. 2 A) or the TSTA (FIG. 2 B). Interestingly, when the PSEBC promoter was inserted as the driver of Cre recombinase, it could titrate DHT levels and the overall system luciferase activity in two AR positive PCa cell lines (22Rv1, LAPC4) but not in AR negative DU145 (PCa) and SW-780 (Bladder cancer) cells (FIG. 2 A).

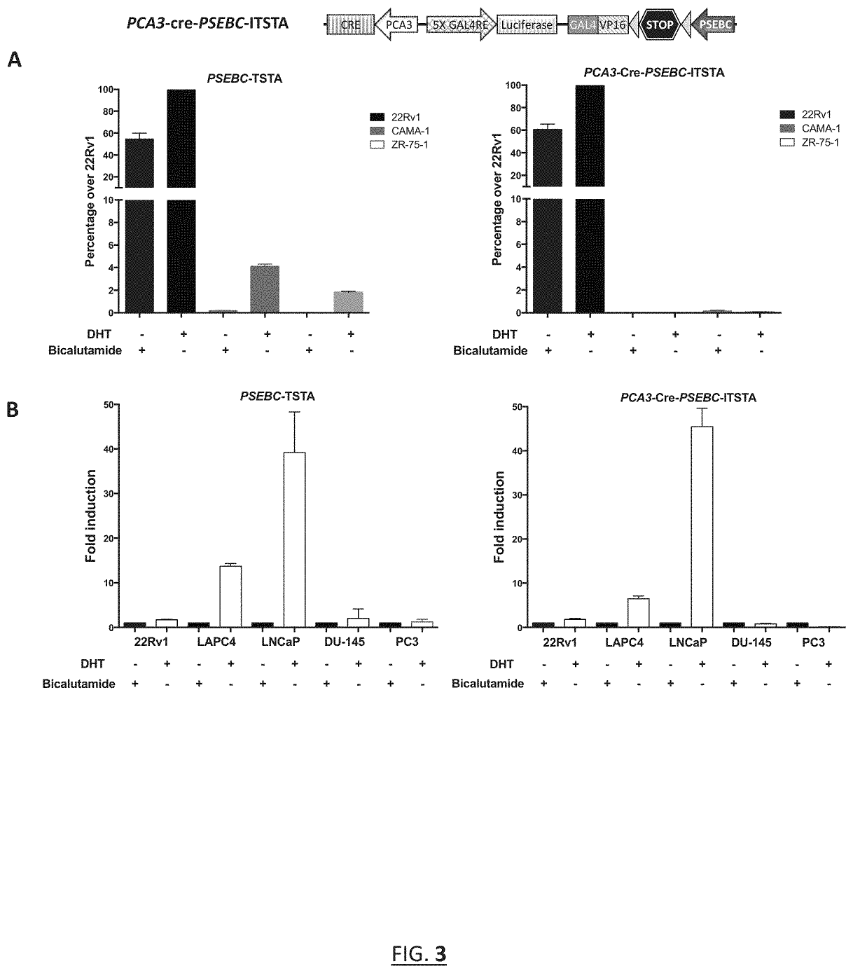

The ability of PCA3-Cre-PSEBC-ITSTA to signal specifically in PCa cells was also tested. PCA3-Cre-PSEBC-ITSTA generated fl activity 1762-times higher in 22Rv1 PCa cells when compared to non-prostatic CAMA-1 cells. Interestingly, this ratio was only 24 when the PSEBC-TSTA system was used (FIG. 3 A, B). In fact, PCA3-Cre-PSEBC-ITSTA not only restricted the expression of fl to PCa cells but also kept the sensitivity of the system to the AR agonists DHT at levels comparable to that obtained when using the PSEBC promoter alone (FIG. 3 A). Also, importantly, PCA3-Cre-PSEBC-ITSTA was not active in AR negative PCa cells (DU-145 and PC3), showing the androgen dependence of the system (FIG. 3 B).

Time Course Study

Because the MP-ITSTA system is a four-step amplification system and because its purpose is to image primary prostate cancer cells over a short period of time in culture, the dynamics for loxP recombination and reporter gene expression had to be determined. Therefore, a time course study comparing PCA3-Cre-PSEBC-ITSTA activity with that of the PCA3-TSTA system was performed. As shown in (FIG. 2 B), fl activity was detectable 24 h after virus infection for both systems and it increased for up to 96 hours after which, a plateau was reached. Interestingly, despite harbouring a more complex system, the PCA3-Cre-PSEBC-ITSTA activity was higher than that of PCA3-TSTA, probably as a consequence of the weak activity of the PCA3 promoter when compared to the PSEBC promoter (Neveu, 2016).

Cre Recombinase Levels Under the Control of PCA3 Promoter

Furthermore, it was determined that the Cre recombinase levels produced by PCA3 weak promoter was sufficient to cleave all the loxP sites. Indeed, quantitative PCR using primer set within the stop cassette or luciferase genes as an internal control showed that PCA3 driven Cre recombinase could cleave up to 95% of the stop cassette after only 48 hours. As a control, Cre recombinase expression in a virus without loxP sites 5′ of the TSTA did not show any cleavage of the stop cassette and DNA copy number of virus remained stable over time (FIG. 2 C).

Material and Methods

Adenoviral Construction

To obtain a single backbone containing the Cre recombinase and the TSTA system with the stop cassette, pENTR-L1R5 and pENTR-L5R2 backbone plasm ids described below were subcloned into pAd-pL-DEST by LR cloning with LRII plus clonase (Invitrogen). Adenoviral backbone containing plasmids were transfected into 293A cells for the viral production. Amplified virus particles were column purified using Adeno-X™ Maxi purification kit (clontech) and stored in buffer A195 after buffer exchange. Titration for each of the viruses was done using Adeno-X™ Rapid Titer Kit (Clontech).

Cell Culture

22Rv1, LNCaP, LNCaP-LN3, LNCaP-PRO5 (prostate cancer cell line) and CAMA-1 (breast cancer cell line), were cultured in RPMI media containing 10% fetal bovine serum (FBS). LAPC4 (prostate cancer cell line) were cultured in DMEM media containing 10% FBS. PC-3 and DU145 (prostate cancer cell lines), SW-780 (bladder cancer cell line) and MD-MB-231, MCF-7 (breast cancer cell lines) were cultured in eMEM media containing 10% FBS. This study was approved by the Institutional Review Board of the CHU de Quebec Hospital, Quebec, QC, Canada. Each patient signed an informed consent form. Cells isolated from patient urine were cultured in RPMI media containing 10% charcoal stripped fetal bovine serum (FBS-CT).

Transfection Experiments

LAPC4 (1.0×10⁵/well) were seeded in 24-well plates. 100 ng of each plasmid was transfected into the cells along with 60 ng of pRL-null (Promega) using lipofectamine 2000 (Lifes technologies). Cells were lysed at the indicated time point using passive lysis buffer (Promega) and luciferase assay was performed as described (Promega).

Adenoviral Infection and Treatment Experiments

22Rv1 (1.0×10⁵/well), LAPC4 (1.0×10⁵/well), LNCaP (1×10⁵/well), LNCaP-LN3 (1×10⁵/well), LNCaP-PRO5 (1×10⁵/well), PC-3 (8×10⁴/well), DU145 (8×10⁴/well), SW780 (8×10⁴/well), CAMA-1 (5×10⁴/well), ZR-75 (5×10⁴/well), MD-MB-231 (5×10⁴/well) and MCF-7 (5×10⁴/well), were seeded in 24-well plates. Twenty-four hours later indicated adenoviruses were transduced at a MOI of 2. Seventy-two hours after infection, cells were lysed and a luciferase assay was performed as described (Promega). For androgen sensitivity assessment, cells were treated with dihydrotestosterone (DHT) at 10 nM or Bicalutamide at 10 μM (Sigma-Aldrich, St. Louis, Mo., USA) or Enzalutamide at 10 μM (MedChem Express, NJ, USA) in 10% charcoal-stripped FBS, 24 h post-infection. Luciferase assays were performed after 48 h of treatment.

Generation of Stop Cassette Plasmids

Bovine growth hormone polyA (Goodwin, 1992) sequence along with the loxP on its flanking ends was synthesised from GenScript (Piscataway, N.J., USA) and the SV40 stop cassette was obtained from plasmid pBS302 (Addgene plasmid 11925) (Kaczmarczyk, 2001). Stop cassette along with the loxP sites at its ends were inserted into pGL3-promoter vector (Promega, Madison, Wis., USA).

PCa cells (LAPC4) were co-transfected with plasmid pBS185 and pENTR-SV40-BGHstop-fl or pENTR-SV40-SV40stop-fl along with pGL3-renilla-null. Forty-eight hours after transfection the cells were lysed and luciferase assay was done. Relative luciferase activity was normalised over renilla. RLU=(firefly luciferase activity/renilla null activity).

Generation of Modified Cre Recombinase

Cre recombinase gene was amplified from plasmid pMC-creN in two fragments: 1-465 bp was amplified using forward primer GGGGAGATTTGTGTGGGTCGACACCATGCCCAAGAAG (SEQ ID NO: 9) and reverse primer GTAACCTTGATACTTACACCTGGTCGAAATCAGTGCGTTC (SEQ ID NO: 10), 466-1523 bp using forward primer TCGTTCACTCATGGAAAATAGCG (SEQ ID NO: 11) and reverse primer GTACAAGAAAGCTGGGTAAAGCTTGTCCGCCACACCCAG (SEQ ID NO: 12) and the intron (human chimeric intron) forward primer GTAAGTATCAAGGTTACAAGACAGGTTTAAG (SEQ ID NO: 13) and reverse primer CTATTTTCCATGAGTGAACGACTGTGGAGAGAAAGGCAAAGTG (SEQ ID NO: 14) from plasmid pIC (Kaczmarczyk) or intron MPI (mice protamine intron) forward primer AAGTAGAGGGCTGGGCTG (SEQ ID NO: 15) and reverse primer CCATGAGTGAACGAACCTAGAAAGGTAAGAAAAGTG (SEQ ID NO: 16) or intron MPI-AG (mice protamine intron containing an AG splice site) forward primer GTAGAGGGCTGGGCTGGGC (SEQ ID NO: 17) and reverse primer CCATGAGTGAACGACTAGAAAGGTAAGAAAAGTG (SEQ ID NO: 18) from gDNA of mice were amplified in 3 different PCR reactions. Three fragment ligation into the plasmid pENTR-L5R2 backbone was done using Gibson assembly cloning kit (New England Biolabs, Ipswich, Mass., USA).

Prostate cancer cells (LAPC4) were co-transfected with plasmids pENTR-PCA3-Cre (WT, BHG-Ig, Prm2 and Prm2-AG) and pENTR-SV40-stop-GAI4VP16-GAL4RE-fl along with pGL3-renilla-null. Seventy-two hours after transfection the cells were lysed and luciferase assay was done. Relative luciferase activity was normalised over renilla. RLU=(firefly luciferase activity/renilla null activity).

Construction of Plasmid with loxP Sites

To determine the affect of residual loxP site left behind post recombination, one loxP was inserted upstream of the GAL4VP16 promoter in the plasmid pENTR-PSEBC-GAL4VP16 (Neveu, 2016). To obtain plasmids containing the TSTA, fl, loxP sites and stop cassette (BGH poly) in a single backbone, SV40 promoter sequence, loxP sites flanking the stop cassette and GAL4VP16 sequence were synthesized (Genscript). Plasmid pENTR-L1R5-GAL4RE-fl (Neveu, 2016) was digested using SalI restriction site and the synthesised fragment was inserted into the plasmid using In-Fusion HD cloning kit (Clontech, Mountain View, Calif., USA) into different orientations shown in supplementary FIG. 4. Prostate specific PSEBC promoter was amplified from pENTR-PSEBC-GAL4VP16 the using forward primer GGATCCGTCGAATTTAAATAAATCTAGCTGATATAGTGTGGC (SEQ ID NO: 19) and reverse primer ATACGAAGTTATTGCGCAGGCTGGGGAGCCTCCCCCAG (SEQ ID NO: 20). SV40 promoter was digested out with SalI and BsiWI restriction sites and replaced by PSEBC using In-Fusion HD cloning kit (clonetech).

Relative Orientation of Each Component of the System

PCa cells (22Rv1, LAPC4, LNCaP, DU-145), breast cancer cells (CAMA-1) and bladder cancer cells (SW-780) were infected with non-replicative adenovirus with the above-mentioned orientations at 2 MOI. Seventy-two hours after infection the cells were lysed and luciferase assay was performed. Relative luciferase activity was normalised over total protein and then normalised over luciferase activity driven by SV40 promoter in each cell line. (relative activity=(RLU/μg protein)÷(RLU SV40/μg). Each data represents triplicates±SD.

Specificity of the System

Prostate Cancer (22Rv1, LAPC4 and DU-145), Bladder cancer (SW-780) and Breast cancer (CAMA-1) cells were infected with replication deficient adenovirus expressing the firefly luciferase gene under control of PSEBC-Cre-SV40-TSTA-fl or PSEBC-Cre-PCA3-TSTA-fl at 2 MOI. Twenty-hours later cells were treated with increasing concentrations of DHT (0-100 nM). Forty-eight hours later cells were lysed and luciferase assay was done.

AR responsive (22Rv1, LAPC4, LNCaP), AR non-responsive (DU-145 and PC3) and AR responsive breast cancer (ZR-75 and CAMA-1) cells were infected with replication deficient adenovirus expressing the firefly luciferase gene under control of PSEBC-TSTA or PCA3-Cre-PSEBC-fl at 2 MOI. Twenty-four hours later media was changed to add DHT (10 nM). Forty-eight hours after treatment cells were lysed and luciferase activity was measured. The relative luciferase activity was first normalized by protein content in each well and then normalized according to the average of luciferase activity driven by SV40 promoter in each cell line (relative activity=(RLU/μg protein)÷(RLU SV40/μg) and represented as relative activity over 22Rv1 in the case of (A) or relative activity over bicalutamide in the case of (B). Each data represents triplicates±S.D.

Time Course Study

22Rv1 cells were infected with the PCA3-Cre-PSEBC-TSTA and PCA3-TSTA adenoviruses and were harvested. At the indicated time points (0, 6, 12, 24, 48, 72, 96, 120 hour), luciferase assay was done.

Cre Recombinase Levels Under the Control of PCA3 Promoter (RT-qPCR Technique)

22Rv1 cells were infected with replication deficient adenovirus containing Cre recombinase and without Cre as control. Cells were harvested and washed with PBS and trypsinized at each of the time points (6, 24, 48, 72, 96 hour). Viral DNA was isolated from the infected cells using QIAmp viral DNA isolation kit (QIAGEN). RT-qPCR reaction was performed for the isolated DNA with taqman probes using two primer sets, one within the stop cassette to determine the cleavage (forward primer TACGGCTGATCAGCCTCGACT (SEQ ID NO: 21), reverse primer AATGACACCTACTCAGACAATGCGAT (SEQ ID NO: 22) and probe TGCCCCTCCCCCGTGCCTTCCTTGA (SEQ ID NO: 23) and the second primer set within the fl gene used as internal control (forward primer ATGGTGGCTTTACCAACAGTACCG (SEQ ID NO: 24), reverse primer TATGAAGAGATACGCCCTGGTTCCT (SEQ ID NO: 25) and probe AGGCCCGGCGCCATTCTATCCGCTGGA (SEQ ID NO: 26)). Standard curves for both the primer sets were determined using plasmids pAd-PCA3-Cre-PSEBC-TSTA-fl or pAd-PCA3-GFP-PSEBC-TSTA-fl as the template. Isolated viral DNA copy number at each time point was extrapolated on the standard curves. PCR cycle used for first primer set was 50° C. for 2 mins, 95° C. for 10 mins, 95° C. for 15 secs and 56° C. for 1 min and for second primer set was 50° C. for 2 mins, 95° C. for 10 mins, 95° C. for 15 secs and 58° C. for 1 min.

Relative copy number (RCN)=((copy number of stop cassette/copy number of luciferase)/RCN at 6 hour×100). Relative luciferase activity was normalised over total protein and then normalized according to the average of luciferase activity driven by SV40 promoter in each cell line. (RLU)=((Firefly luciferase activity/total protein)/SV40 activity). Each data represents triplicates±SD.

### Example 2—Bioluminescence Microscopy as a Method to Measure Single Cell Androgen Receptor Activity Heterogeneous Responses to Antiandrogens

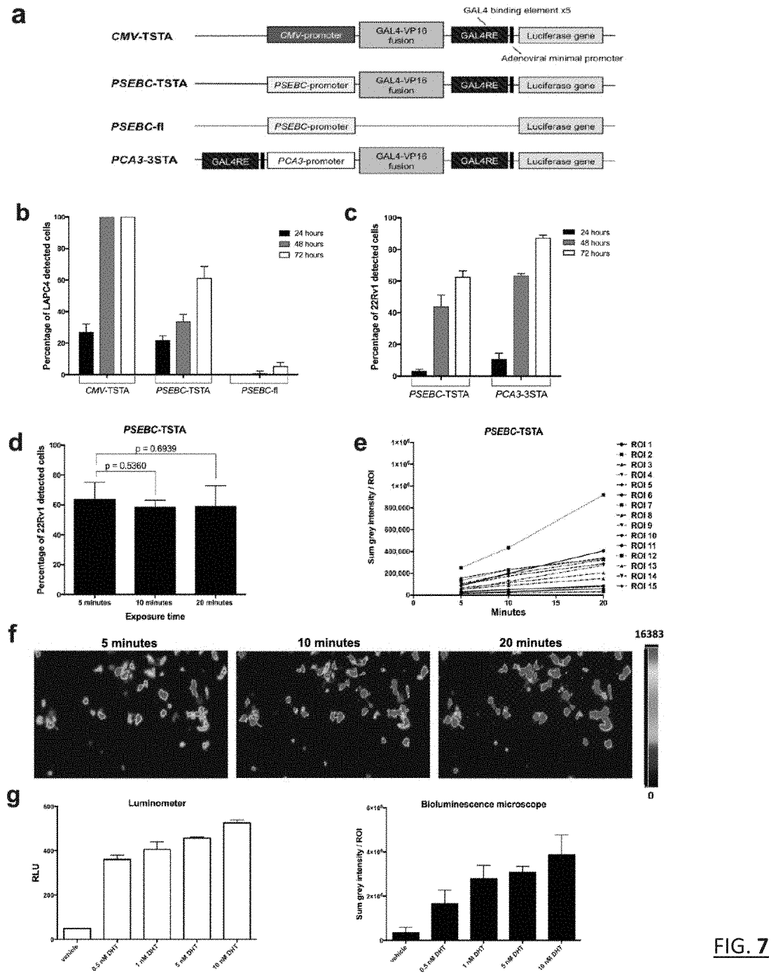

With the goal of imaging primary prostate cancer (PCa) single cell response to antiandrogens, the first step was to develop conditions for an appropriate imaging system driven by a promoter containing the androgen response elements sequence (ARE), which could be delivered into PCa cells. Because of high infectivity and thorough characterization in primary PCa cells, type 5 adenovirus was chosen as the delivery method (Neveu, 2016). For the PCa cell imaging using bioluminescence microscopy, the following constructs were created; type 5-adenovirus-enabling firefly luciferase (fl) expression driven by either a strong ubiquitous promoter (CMV), a well-characterized ARE-bearing promoter (PSEBC) (Pouliot, 2009) (Wu, 2001), or a PCa-specific androgen-insensitive promoter (PCA3) (FIG. 7 A) (Neveu, 2016).

Optimization of the Fl Substrate, D-Luciferin

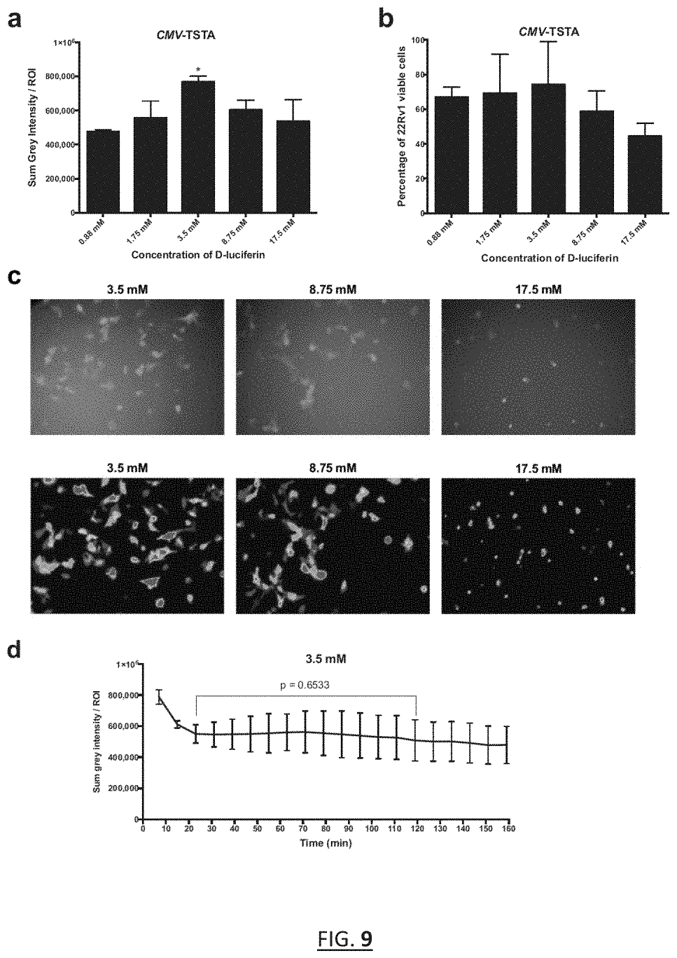

Using a transcriptional amplification system (TSTA) and a CMV promoter, a test was performed to establish whether increasing D-luciferin concentration could enhance fl activity per region of interest (ROI). As shown in (FIG. 9 A), optimal ROI sum grey intensity in 22Rv1 was achieved at a concentration of 3.5 mM of D-luciferin. When the D-luciferin concentration was increased up to 17.5 mM, the overall fl activity decreased by 30%, most likely secondary to cell toxicity (viability decreased to 40% with the highest dose (FIG. 9 A, C)).

Optimization of the Exposure Time for Signal Intensity

Because some dynamic bioluminescence studies would involve multi-well (many wells at the same time) and multi-condition (such as different exposure times) imaging, the signal sustainability over time following substrate exposure had to be determined. When fl activity was quantified over time following CMV-TSTA transduction, a sustained luciferase signal with no significant reduction from 20 min to 120 min following the addition of 3.5 mM of D-luciferin was detected (FIG. 7 D).

Optimization of the Conditions for Viral Transduction

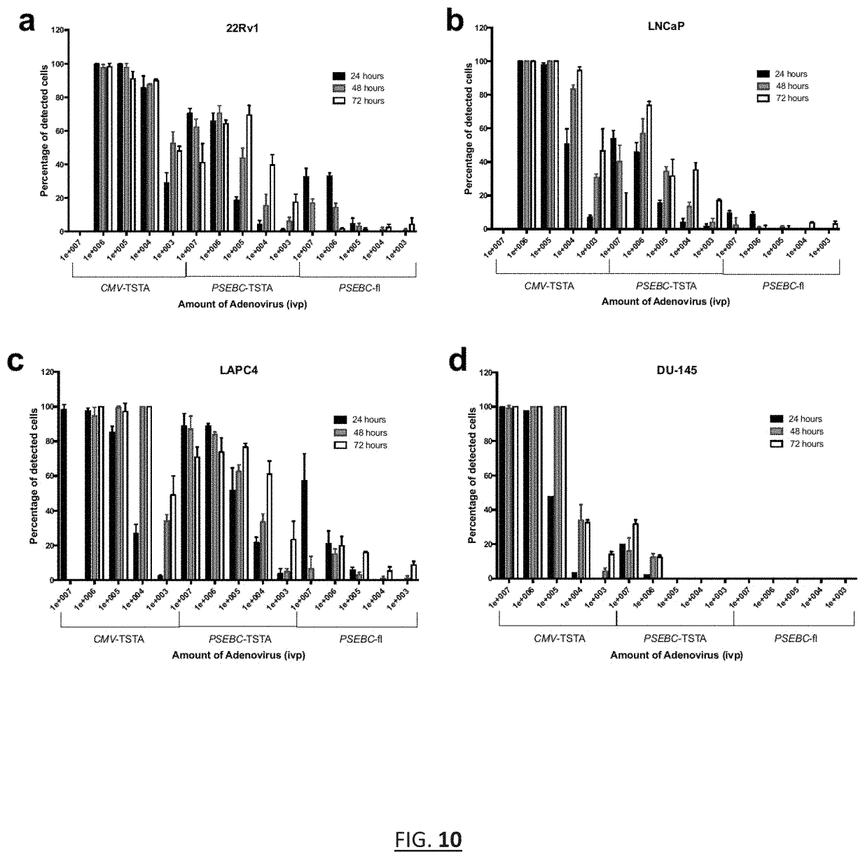

Another optimization step was to establish the optimal conditions for viral transduction and the amount of infectious viral particles (ivp) required to enable the detection of more than 90% of the cells, as the ultimate goal was to enable the detection of primary PCa cells in fluid biopsies (FIG. 7 B and FIG. 10). As shown in (FIG. 7 B) and (FIG. 10), upon testing various amounts of infectious viral particles (10³-10⁷ ivp), 10⁴ ivp of CMV-TSTA detected more than 90% of the cells in 3 out of the 4 PCa cell line populations tested, except DU-145 (AR−) PCa cells, thereby confirming this amount of adenovirus was appropriate for optimal transduction. However, in the DU-145 cell line, 90% of the cells were detected only with higher amounts (10⁵ ivp, FIG. 10 D). When the androgen-responsive PSEBC-fl adenovirus was tested at higher amount (10⁷ ivp), only 31, 10, and 57% of the cells were detected in 22Rv1, LNCaP, and LAPC4 cell lines, respectively, although cell viability was greatly affected at this amount (FIG. 10 A, C). Therefore, to increase fl reporter gene expression and detection rates, the PSEBC promoter was cloned in the TSTA system to generate the PSEBC-TSTA (Pouliot, 2009) (FIG. 7 A). PSEBC-TSTA detected more cells than PSEBC-fl virus did at 10⁵ ivp but much less than CMV-TSTA recorded. Only 73, 43, and 76% of the cells were detected in the 22Rv1, LNCaP, and LAPC4 cell lines, respectively (FIG. 10 A, C). As expected, no expression was observed in DU-145 (AR−) after 72 h of viral infection (FIG. 10 D). Interestingly, when the PCa-specific and androgen-insensitive PCA3-3STA (Neveu, 2016) promoter systems were used for cell imaging, the percentage of detected PCa cells reached that of the CMV-TSTA (FIG. 7 C).

Effect of the Exposure Time on the Detection of Cells

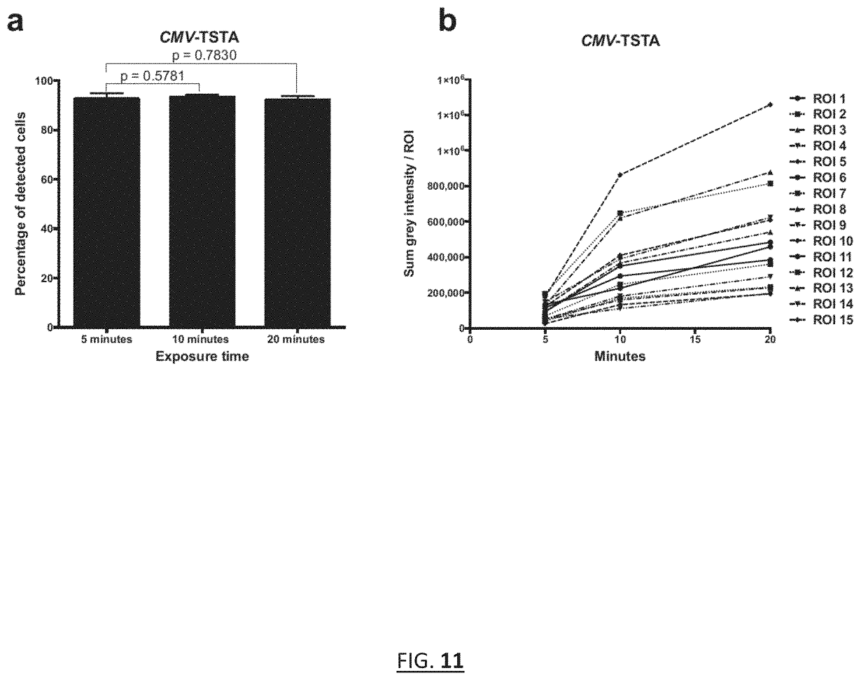

To further exclude confounding factors explaining heterogeneous single cell PSEBC activity within AR+cell lines, an analysis was performed to determine whether exposure time could impact the number of detected cells. FIG. 7 D, F and FIG. 11 show that prolonging exposure time by 4-fold did not enhance the percentage of detected cells using either the CMV-TSTA (FIG. 11 A) or PSEBC-TSTA (FIG. 7 D) system. However, increasing the exposure time did increase the sum of activity of each ROI (FIG. 7 E and FIG. 11 B).

Single Cell Heterogeneous Activity in the Same Androgen-Sensitive (AR+) PCa Cell Lines

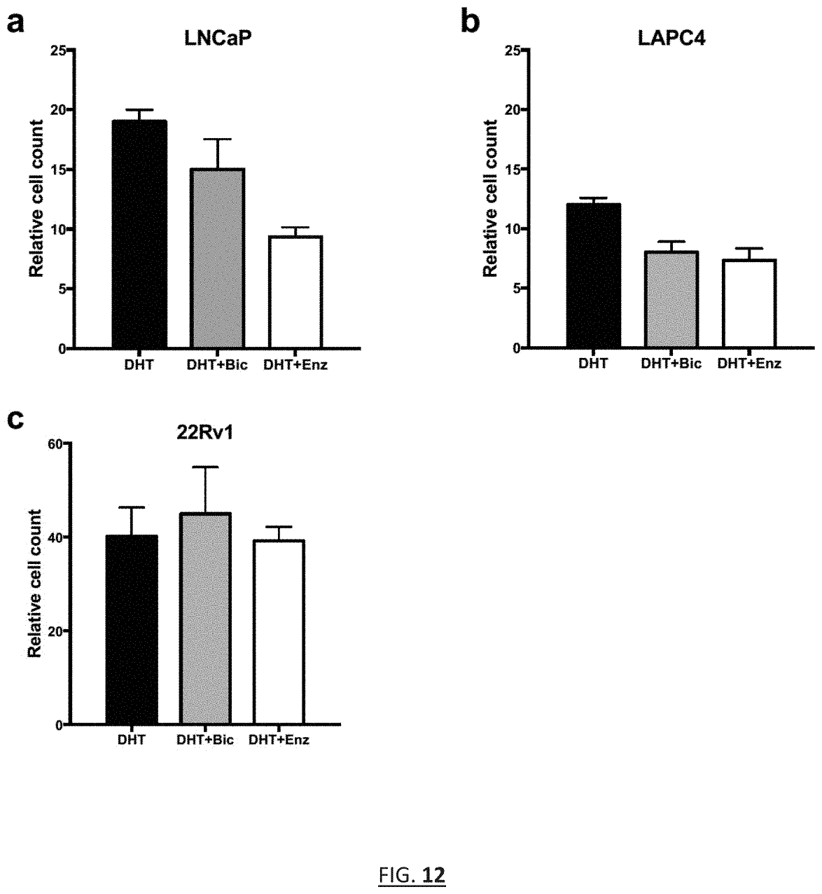

Together, these results show that transduction (FIG. 7 B, C), exposure time (FIG. 7 D, F), or fl level of expression (FIG. 7) could not explain absence of PSEBC activity in around 40% of the cells, depicting single cell heterogeneous activity in the same androgen-sensitive (AR+) PCa cell lines. Thus, it was shown that the PSEBC promoter was inactive in many cells of AR sensitive PCa cell lines, even though the androgen sensitivity of these cell lines as a whole remained the same (FIG. 12 A, C). This showed that PSEBC-TSTA had the ability to specifically detect androgen-sensitive cells and there is a hidden androgen-insensitive population within the same AR+PCa cell line.

Bioluminescence Microscopy is Quantitative and Suited to Measure Single Cell AR Activity Modulation by Antiandrogens

Imaging single cells with increasing exposure times allowed the observation of a linear increase in the grey intensity with a mean activity ratio of 1.79- and 3.20-fold between 5 to 10 min and 5 to 20 min, respectively (FIG. 7 E, F), depicting the high accuracy of bioluminescence microscopy following adenoviral transduction (y=13961x−7680, r²=0.9763).

Comparison Between Luminometer and Bioluminescence Microscopy

Enzalutamide (Enz) is a novel highly potent second generation-AR antagonist indicated for castration-resistant PCa, while bicalutamide (Bic) is a weaker, classical AR antagonist used in the early-stages of the disease.

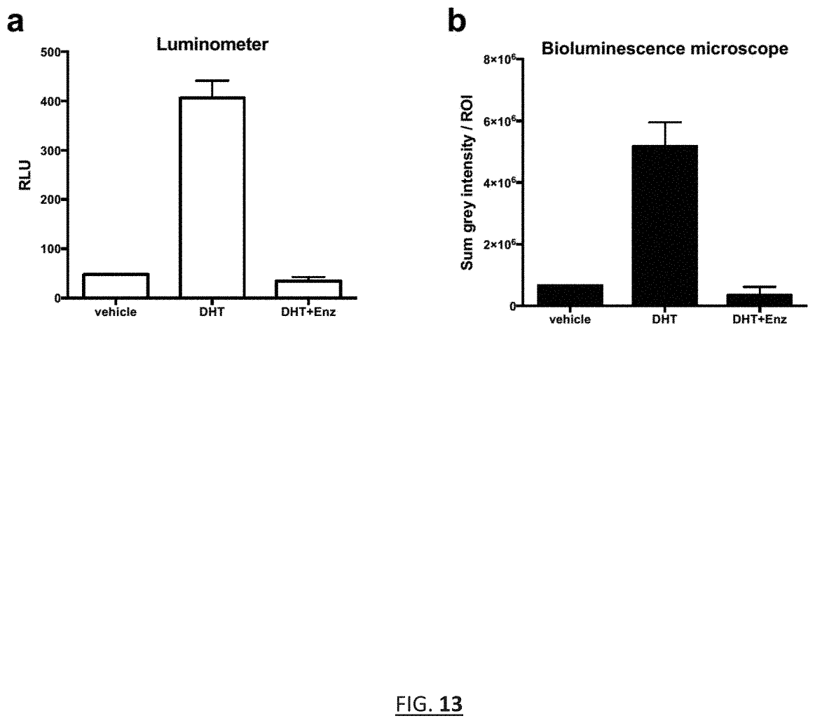

To further evaluate how the method could quantify AR activity modulation by androgens and antiandrogens, it was compared to the current gold standard used for bioluminescence quantification, namely luciferase assays on whole cell lysates using a luminometer apparatus (McNabb, 2005). A comparison between luminometer and bioluminescence microscopy quantifications was conducted in LAPC4 cells infected with PSEBC-TSTA and exposed to vehicle (ethanol), DHT, or DHT+Enzalutamide (FIG. 13). As expected, following luminometer quantification, the normalized RLU was induced by DHT, an effect that was completely inhibited by enzalutamide (8.4±0.65 and 0.72±0.17-fold, respectively). Similarly, luminescence microscopy measured AR induction by DHT or complete inhibition by enzalutamide (7.80±0.85 and 0.52±0.18-fold, respectively). Because DHT is a direct agonist of AR, the ability of bioluminescence to titrate DHT concentrations was measured (FIG. 7 G). Again, bioluminescence measurements were linear over increasing doses of DHT (y=0.044x+1.03, r²=0.9512) (FIG. 7 G). This linear coefficient was similar to that obtained with the luminometer (y=0.11x+1.26, r²=0.77) (FIG. 7 G). Correlation between luciferase signal and DHT concentration were compared between bioluminescence microscope and luminometer using the Fisher's Z-transformation. This comparison showed no significant difference (Fisher's Z value=0.6844, p=0.4937). Overall results indicate that bioluminescence microscopy was as quantitative as luciferase assays but provided the advantage of enabling single cell activity measures.

Single Cell Analysis of PSEBC-TSTA Infected LAPC4 (AR+) PCa Cells Cultured in DHT Containing Media with or without Antiandrogens

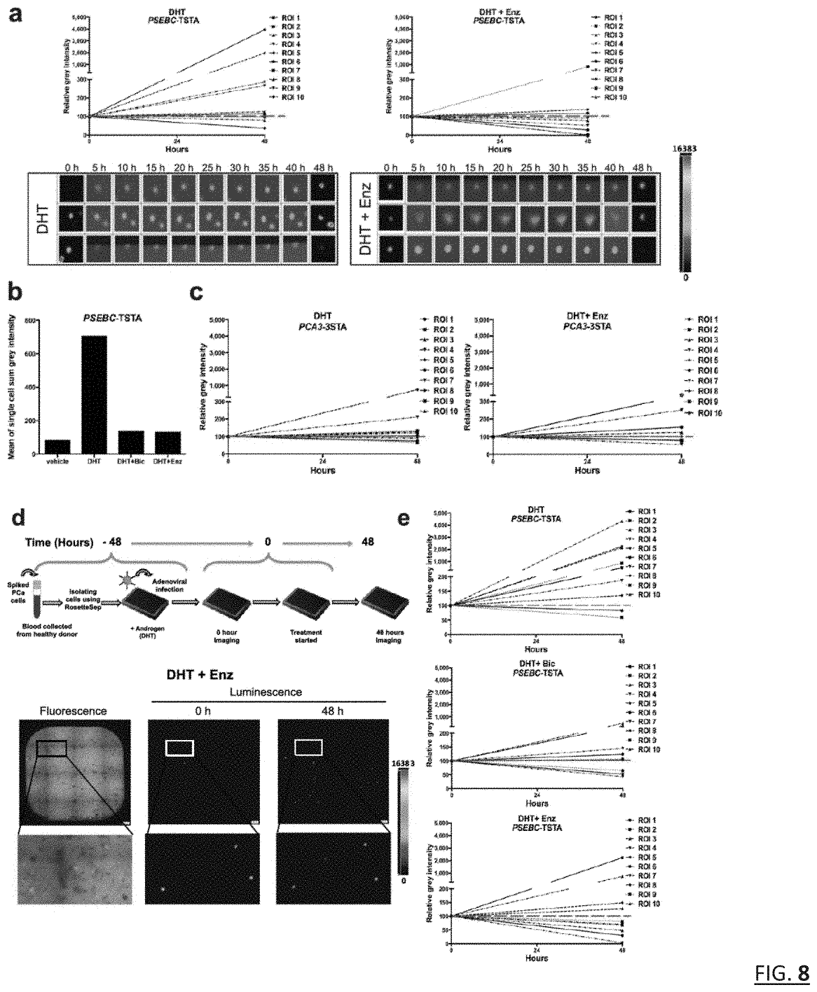

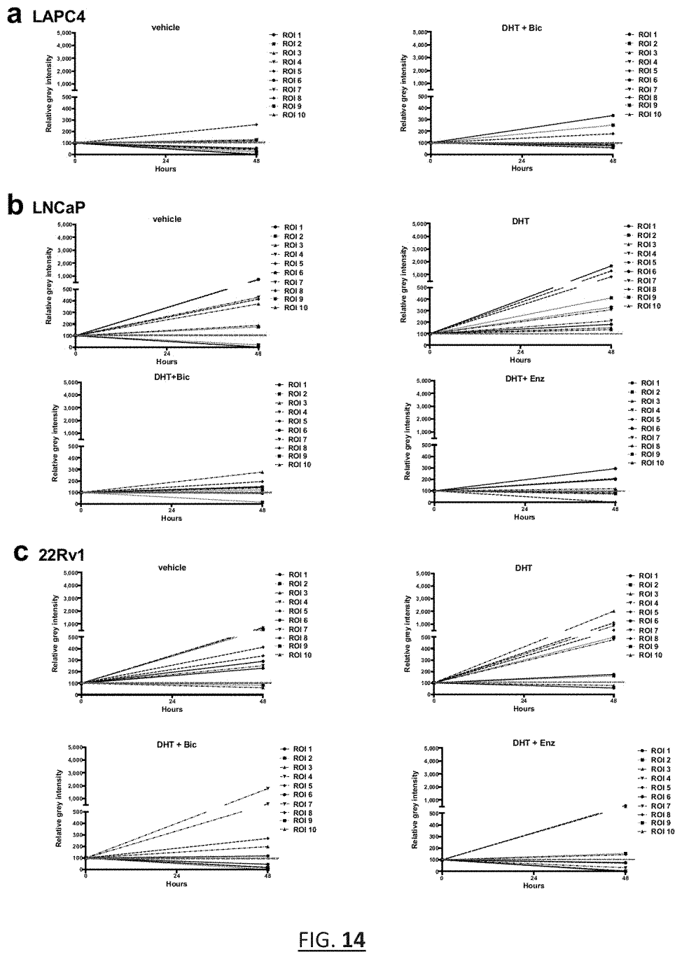

To exploit the unique ability of the microscope to quantify cell-by-cell luminescence using an androgen-modulated PSEBC promoter, LAPC4 (AR+) PCa cells were cultured in DHT containing media with or without antiandrogens. Quantification of the single cell sum grey intensity revealed heterogeneous response patterns to DHT or antiandrogens within the same cell line (FIG. 8 A, more data in FIG. 14). When LAPC4 cells were treated with AR agonist DHT, most of the cells (80%, n=10) displayed an increase in fl activity, while the remaining 20% showed a decrease. In contrast, upon withdrawal of the androgens or under antiandrogen (Enzalutamide) treatment, most of the cells showed a decrease in fl activity (70%), with 30% showing an increase (FIG. 8 A).

Single Cells Analyzed with Bioluminescence Microscopy are Representative of the Overall Cell Line Hormonal Responsiveness

Because single cell imaging relies on isolated cells, there was a need to ensure that the selected cells continued to maintain cell line androgen and antiandrogen responses and that the cells were not, for example, cells with aberrant responses. For each treatment group (Vehicle, DHT, DHT+Bic, DHT+Enz), the sum of fl activity of ten single cells analyzed following PSEBC-TSTA transduction and bioluminescence microscopy quantification (FIG. 8 A) was calculated. As shown in FIG. 8 B, similar to the LAPC4 cell line, the single cells (also LAPC4) were strongly induced by DHT (8.8-fold), an induction inhibited by both Bic and Enz. These results show that single cells sampled and analyzed were representative of the overall cell line hormonal responsiveness and that luminometer luciferase assays represented the sum of a heterogeneous cell population, with some being inhibited and some being induced.

Transcriptional Responses Observed Following Hormonal Treatments are not Due to their Indirect Effect of Transcriptional Inhibition on Cell Viability

To ensure that the transcriptional responses observed following the hormonal treatments of DHT, Enz or Bic were not due to their indirect effect of transcriptional inhibition on cell viability, the method was tested on AR+22Rv1 cells. These cells have a functional AR pathway but are resistant to antiandrogen growth inhibition but may still be modulated by Enz and Bic (FIG. 14 A) (Li, 2015). FIG. 14 C shows that 22Rv1 single cell fl activity was still modulated by both the androgens and antiandrogens, demonstrating that the short-term treatment effect was secondary to the AR transcriptional modulation by AR ligands such as Enz. As an additional control, hormonal treatments were tested on cells transduced with PCA3-3STA, an androgen-insensitive system (FIG. 8 C). It was observed that PCA3 promoter-dependent single cell bioluminescence activity did not show induction nor inhibition on addition of DHT or DHT+Enz. The overall fold change with PCA3 promoter-dependent activity for 10 cells on addition of DHT or DHT+Enz was 1.75- and 1.09-fold, respectively (FIG. 8 C), showing again that the treatment effects observed earlier using PSEBC-TSTA were secondary to the transcriptional modulation by AR ligands.

Translation of the Bioluminescence Microscopy Method to Blood Samples

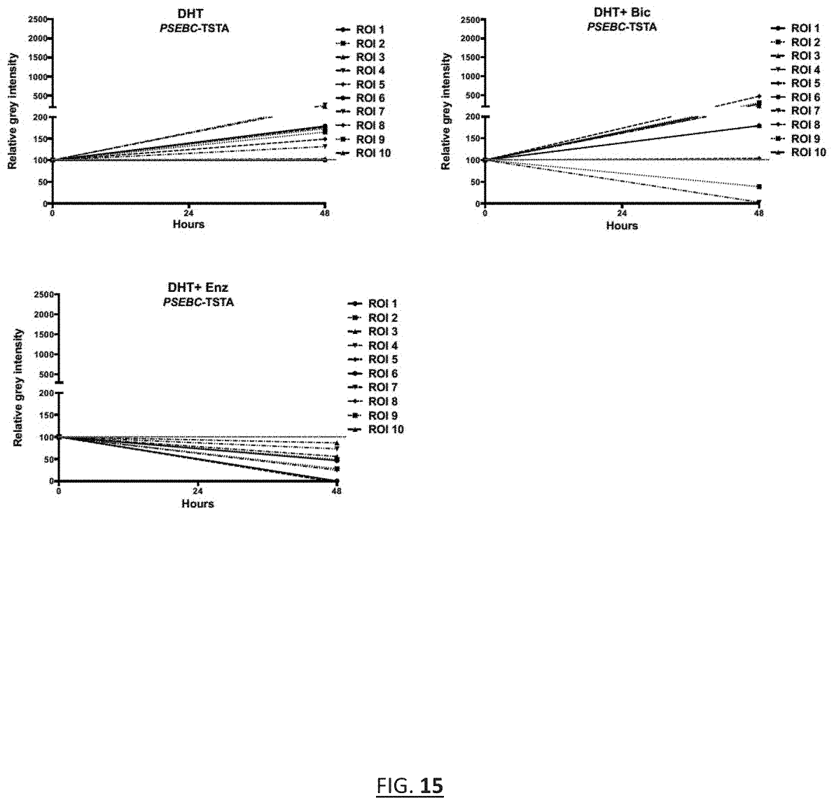

Furthermore, to translate single cell bioluminescence microscopy quantification methods into a clinical application, PSEBC-TSTA was used to target spiked cancer cells isolated from the blood of healthy individuals. Those experiments ensured that this optimized method could image single cells harvested from blood in a heterogeneous cell population. Following the enrichment of blood with cancer cells, cells were transduced with PSEBC-TSTA and cultured for 48 h in the presence of Enz or Bic. As shown in FIG. 8 D, the spiked cells were targeted with the adenovirus (PSEBC-TSTA), which enabled the detection of 60-77% of the cells (luminescent cells over fluorescent cells). In addition, when isolated cells were treated with Bic or Enz, despite the presence of remaining blood cells, it was possible to image the antiandrogen AR transcriptional response with high specificity, as all of the bioluminescent cells (LAPC4 or LNCaP) were also fluorescent (FIG. 8 D, E and FIG. 15). Noticeably, LNCaP cells (which are highly sensitive to Enz) showed a homogeneous response to Enz (FIG. 15), while the response of isolated LAPC4 cells was highly variable (FIG. 8 E).

Material and Methods

Plasmid Construction and Adenoviral Production

Adenoviral plasmids for PSEBC-TSTA, PCA3-3STA, and CMV-TSTA were constructed as previously described 18. PSEBC-fl was devised using gateway cloning for adenoviral constructs. The PSEBC promoter was PCR-amplified from pENTR-L1R5-PSEBC-GAL4VP16 and inserted into a pENTR-L1R2 backbone plasmid to build PSEBC-fl. Lentivirus-expressing renilla luciferase as well as GFP were constructed using the plasmid pccl-CMV-RL-IRES-EGFP12. Five p g of pccl-CMV-RL-IRES-EGFP plasmid and three helper plasmids (Gag-Pol, Rev and VSV-G) were transfected into 293T cells using lipofectamine 2000 (Life Technologies, Burlington, ON, Canada), in a 60 mm Petri dish. Virus particles were collected thereafter and titrated using serial dilutions and GFP-positive cells were counted by means of fluorescent activated cell sorting (FACS).

Cell Cultures

22Rv1 and LNCaP (prostate cancer cell lines) were cultured in RPMI-1640 media containing 10% fetal bovine serum (FBS). LAPC4 (prostate cancer cell line) and HEK 293 (human embryonic kidney cells) were cultured in DMEM media containing 10% FBS. DU-145 (prostate cancer cell line) was cultured in EMEM media containing 10% FBS. LAPC4 and DU-145 were kindly provided by Dr. C. Sawyers and Dr. L. Old respectively. Other cell lines were obtained from ATCC. The cell lines were tested for absence of mycoplasma using the MycoAlert Mycoplasma Detection kit (Lonza, Basel, Switzerland).

Production of Stable Transduced Cell Lines

22Rv1, LAPC4, LNCaP, and DU-145 were seeded in a 24-well plate (10,000 cells/well). Twenty-four hours later, the lentivirus was transduced at a multiplicity of infection (MOI) of 5 along with polybrene (8 μg/ml). Twenty-four hours post-infection, the media was changed to remove the virus and the cells were kept in culture until more than 60% of GFP expressing cells was obtained. The cells were then trypsinized and collected in PBS containing 2% FBS. GFP-positive cells were sorted by means of FACS to obtain 22Rv1-GFP, LAPC4-GFP, LNCaP-GFP and DU-145-GFP. These cells were maintained in culture for one passage to propagate before using them in experiments.

Bioluminescence Microscopy

Bioluminescence imaging was performed using an Olympus LV200 microscope equipped for luminescence imaging, transmitted brightfield and transmitted fluorescence imaging. Samples to be imaged were seeded in a 384-well black plate (Ibidi, Madison, Wis., USA) and placed on a motorized stage (Prior Scientific, Rockland, Mass., USA) provided with a stage-top incubator (Tokai Hit, Fujinomiya, Japan). Luminescence imaging was performed using either 20× (5 min of exposure per field of view (FOV)) or 40× (15 s of exposure per FOV) objectives. The emitted photons passed through an open channel without filter and collected onto an electron-multiplying CCD camera (Andor Ixon 897). For the biofluorescence imaging, samples were excited at a wavelength of 470 nm (X-Cite XLED1, Excelitas Technologies, City, MA, USA) and the fluorescence emission of eGFP was collected using the CCD camera. Data analysis and process design for automated image capture were achieved using the cellSens software (Olympus, Tokyo, Japan).

Adenoviral Infection and Viability Assay

22Rv1, LAPC4, LNCaP, and DU-145 cells (1,000 cells/well) were seeded in a 384-well black plate. Twenty-four hours later, the cells were transduced with either CMV-TSTA, PSEBC-TSTA, PSEBC-fl or PCA3-3STA adenovirus. Imaging was performed with 3.5 mM of D-luciferin (re-suspended in PBS) (Caliper Lifesciences, Hopkinton, Mass., USA). Media was refreshed at each time point (24, 48, and 72 h) and imaging was done with an exposure of 5 min per FOV by means of the LV200 bioluminescence microscope. The percentage of detected cells was defined as the number of bioluminescent over biofluorescent cells (GFP-positive cells) multiplied by 100. At the end of the protocol, following imaging, 5 μl of PrestoBlue reagent (ThermoFisher Scientific, Waltham, ON, Canada) was added to each well in 45 μl of media and incubated overnight at 37° C. in a cell culture chamber. The media was then collected and fluorescence was measured by Fluoroskan Ascent (ThermoFisher Scientific) at the excitation/emission wavelength of 540/595 nm. Cell viability was defined as follows: (fluorescence of infected cells−fluorescence media)÷(fluorescence control non-infected cells−fluorescence media)×100.

D-Luciferin Concentration Optimization

22Rv1 cells were seeded in a 384-well black plate and 10⁴ infectious viral particles (ivp) of CMV-TSTA adenovirus were added 24 h later. Seventy-two hours after infection, 0.88, 1.75, 3.5, 8.75, and 17.5 mM of D-luciferin were added into separate wells and the sum grey intensity was recorded thereafter every 5 min for 2.5 h using the time lapse registering protocol of the cellSens software. Sum grey intensity was first quantified for bioluminescence-positive cells at each concentration in a defined ROI and then normalized by the number of luciferase-positive cells. The viability assay was performed at 72 h only, following the imaging.

Exposure Time Optimization

22Rv1 cells were seeded in a 384-well black plate and either CMV-TSTA or PSEBC-TSTA adenovirus was added 24 h later. Seventy-two hours after infection, D-luciferin (3.5 mM) was added to each well and after 20 min, imaging was performed with exposures of 5, 10 and 20 min in a defined frame. The percentage of detected cells was calculated as described above. Sum grey intensity was quantified using the cellSens software in the same ROI at different exposure times.

Androgen Responsiveness Assessment by Luciferase Assay

22Rv1, LAPC4, and LNCaP cells were seeded in 24-well plates. Twenty-four hours later, 10⁴ ivp PSEBC-TSTA adenovirus was diluted in 50 μl of media containing 10% charcoal-stripped FBS (FBS-CT) and treated with either vehicle (Ethanol), dihydrotestosterone (DHT) (0.5-10 nM, as indicated), DHT+Bic (1 nM+10 μM) (Sigma-Aldrich, St. Louis, Mo., USA), or DHT+Enz (1 nM+10 μM) (MedChem Express, South Brunswick, N.J., USA). Forty-eight hours after treatment, bioluminescence microscopy or luciferase assays were used to measure luciferase activity. For the luciferase assays, the cells were lysed using a passive lysis buffer (Promega, Madison, Wis., USA) and luciferase activity was measured by means of Luminoskan Ascent (ThermoFisher Scientific) following the addition of D-luciferin, as stated in the Dual-luciferase protocol (Promega). Relative fl activity (RLU) was normalized by protein content in each well (RLU=RLU÷μg of protein). Protein content was estimated by adding 250 μl of Bradford reagent (ThermoFisher Scientific) to 3 μl of total lysate. Absorbance was then read using an Infinite F50 absorbance microplate reader (TECAN, Mannedorf, Switzerland) at 595 nm. For the bioluminescence microscopy, 3.5 mM of D-luciferin in fresh media was added to each well and imaging was performed with an exposure time of 2 min per FOV. Sum grey intensity was normalized by the total number of counted fl-expressing cells (Sum grey intensity=sum grey intensity per ROI÷number of fl-positive cells). Sum grey intensity was calculated using the cellSens software.

Single Cell Treatment Response

22Rv1, LAPC4, LNCaP, and DU-145 cells were seeded in a 384-well black plate. Twenty-four hours later, the cells were transduced with 10⁴ infectious viral particles of PSEBC-TSTA or PCA3-3STA in media containing 1 nM of DHT and 10% FBS-CT. Forty-eight hours after infection, the cells were imaged by bioluminescence microscopy and after the treatment was added (vehicle, DHT (1 nM), DHT+Bic (1 nM+10 μM) or DHT+Enz (1 nM+10 μM)). To track the change in position and growth of single cells in which fl activity was measured, biofluorescence microscopy imaging was performed every 5 h for 48 h (GFP-positive cells) after which time bioluminescence imaging and quantification were repeated on the same cells. Sum grey intensity was then determined for single cells before and after treatment using the cellSens soft-ware. Relative grey intensity=((sum grey intensity per ROI−sum grey intensity of background)÷sum grey intensity at 0 h)×100.

Single Cell Treatment Response from Spiked Cancer Cells Isolated from Blood

Blood was collected from a healthy donor and placed in a heparin-coated tube. This study was approved by the Institutional Review Board of the CHU de Quebec Hospital, Quebec, QC, Canada. Informed consent was obtained by the donor for blood sampling. All experiments were performed in accordance with relevant guidelines and regulations. LAPC4 and LNCaP cells were added to the blood sample (500 cells/3 ml of blood), followed by a custom-made RosetteSep cocktail (STEMCELL Technologies, Vancouver, BC, Canada) at a volume of 50 μl per ml. The sample was gently mixed and incubated thereafter at room temperature for 20 min. Fifteen milliliters of Ficoll-Paque Plus (GE healthcare Life Sciences, Mississauga, ON, Canada) were then placed in a 50 ml SepMate tube (STEMCELL Technologies) 31 and blood was poured gently along the walls of the tube onto the Ficoll layer. The tubes were subsequently centrifuged at 1200 g for 20 min at room temperature with the brake on. The top 10 ml of the top layer was removed and the remaining top layer was gently transferred to clean 50 ml tubes. Forty milliliters of PBS containing 2% FBS were then added and the tubes were centrifuged at 350 g for 8 min with the brake on. The supernatant was then gently removed, leaving behind 1 ml in each tube. The pellets were then resuspended in 5 ml of RPMI-1640 media containing 10% FBS-CT and DHT (1 nM) and the tubes were centrifuged at 350 g for 8 min with the brake on. The resulting supernatant was then gently removed to reduce the final volume to 50 μl. The recovered cells were then seeded in a 384-well plate. Cells were transduced with 5×10⁴ viral particles of PSEBC-TSTA. The plate was kept in a shaker overnight. Forty-eight hours after infection, bioluminescence imaging was performed and after the treatments were added (vehicle, DHT (1 nM), DHT+Bic (1 nM+10 μM) or DHT+Enz (1 nM+10 μM)). The cells were tracked every 5 h for 48 h using biofluorescence microscopy. Forty-eight hours later, bioluminescence imaging was repeated on the same cells. Percentage of targeted cells=(number of bioluminescence positive cells÷number of GFP positive cells)×100. The sum of grey intensity values was determined for single cells before and after treatment using cellSens software. Relative grey intensity=((sum grey intensity per ROI−sum grey intensity of background)÷sum grey intensity at 0 h)×100.

Statistical Analysis

All of the statistical analyses were conducted using the two-sided t-test with Welch's correction, with (*) indicating p≤0.05. The variance was consistent within each experimental group. The Fisher's Z test was used to compare the correlations.

### Example 3—Bioluminescence Microscopy with the PCA3-Cre-PSEBC-ITSTA System can Detect PCa Cells from Urine and Quantify Single Cell Response to Anti-Androgen Therapy

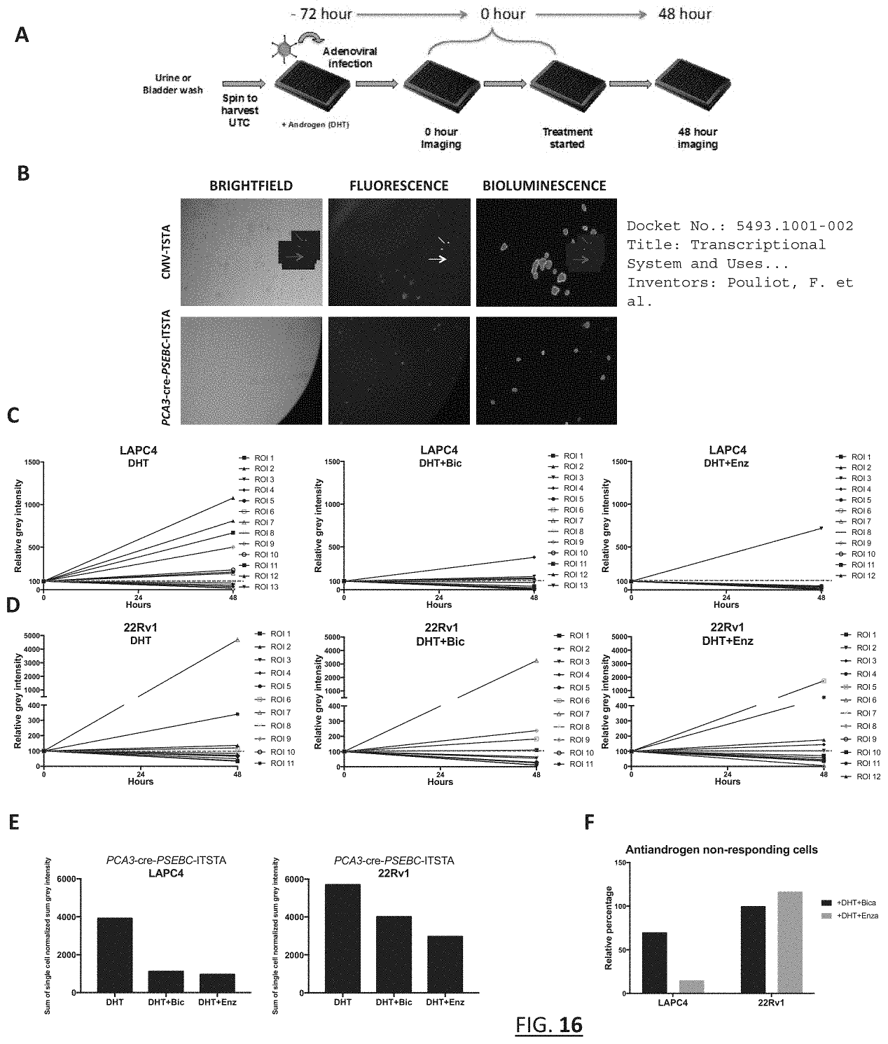

As another step towards clinical translation, the ability of the PCA3-Cre-PSEBC-ITSTA system to detect PCa cells from urine samples was tested (FIG. 16 A).

Spiked Cell Recovery and Transduction with the PCA3-Cre-PSEBC-ITSTA System into Urine Samples from Healthy Donors

When 22Rv1 cells stably expressing mCherry were spiked into the urine of a patient without prostate or evidence of disease (undetectable PSA after radical prostatectomy), it was seen that 89% of the spiked cells could be recovered (data not shown). After urine cell isolation and transduction with PCA3-Cre-PSEBC-ITSTA, bioluminescence microscopy detected signal only in PCa cells, expressing mCherry. However, when urine cells were transduced with an adenovirus expressing luciferase under an ubiquitous CMV promoter, non-specific expression in non-PCa cells was seen, showing that PCa specificity of PCA3-Cre-PSEBC-ITSTA relied on its promoter activity rather than transduction (FIG. 16 B).

Single Cell Analysis from Urine Samples with Bioluminescence Microscopy is Representative of the Overall Cell Line Hormonal Responsiveness

PCa Enz sensitive (LAPC4-GFP) and resistant (22Rv1-GFP) cells were spiked into urine, isolated, infected with PCA3-Cre-PSEBC-ITSTA and exposed to DHT, DHT+Bic or DHT+Enz treatments. As seen in (FIG. 16 C, D), PSEBC promoter could determine heterogeneous single cell response to androgens and antiandrogens within the same cell population. In case of LAPC4, 40% of cells showed an increase in the luciferase activity under antiandrogen (bicalutamide) treatment while 60% showed a decrease in their activity (FIG. 16 C), thereby showing that PCA3-Cre-PSEBC-ITSTA could determine the single cell response of AR+LAPC4 cells to antiandrogens (FIG. 16 C). After a calculation of the sum of single cell activity in DHT only vs DHT+ anti-androgen groups, a significant difference between the DHT only and both antiandrogen groups was observed (FIG. 16 E).

Transcriptional Responses Observed Following Hormonal Treatments are not Due to their Indirect Effect of Transcriptional Inhibition on Cell Viability

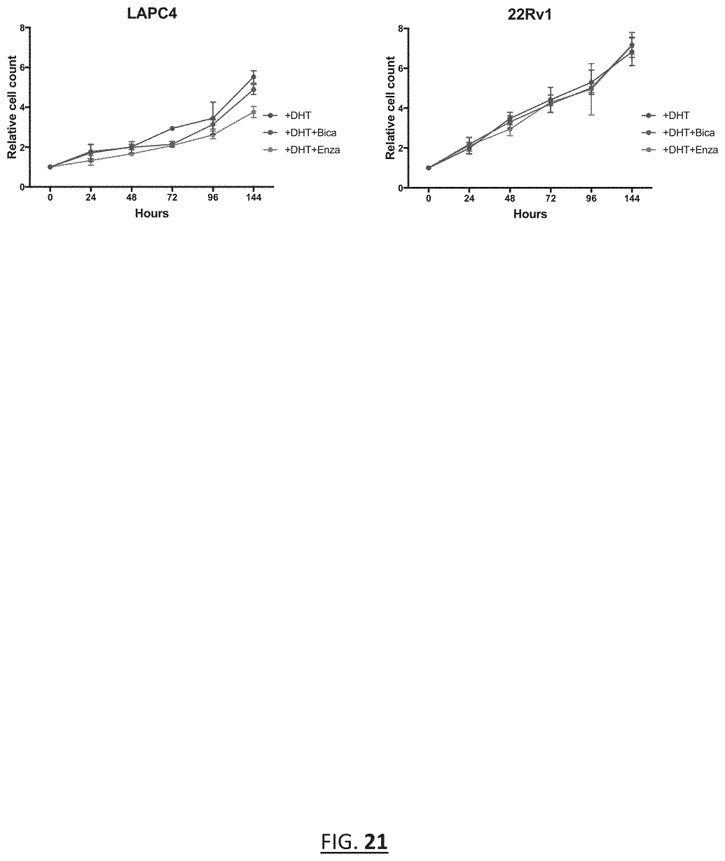

To ensure that the transcriptional responses observed following the hormonal treatments were not due to their indirect effect on cell viability, the method was tested on AR+22Rv1 cells. These cells have a functional AR pathway but are resistant to antiandrogen growth inhibition (FIG. 16 D, E). As for LAPC4 cells, 22Rv1 single cell fl activity was modulated by both the androgens and antiandrogens. Because the sum of single fl activity was modulated between the DHT only and both antiandrogen groups and because 22Rv1 are not growth inhibited by antiandrogens (FIG. 21), these results show that AR-dependent transcriptional modulation by antiandrogen was not secondary to cell death under treatment (FIG. 16 D).

The Proportion of Cell Sensitivity to Antiandrogen is a Better Surrogate of Growth Response than Bulk Cell Population Analysis

However, the modulation of AR-dependent transcription in 22Rv1 did show that the sum of single cell activity was not a good surrogate of cell line antiandrogen sensitivity. Because a decreased luciferase activity in cells over time can be secondary to AR-transcriptional response or non-specific cell death, only the cells with an increase over baseline were considered as a surrogate of overall cell population sensitivity. Thus, for each cell line, the relative percentage of cells showing an increase in luciferase activity over time in the antiandrogen treated cells compared to that of the DHT only treated cells was considered. Cells with a decrease in luciferase activity over time in DHT only treated groups were considered as indicators of non-specific cell-death under culture conditions. Interestingly, the percentage of cells with increased activity in LAPC4 Enz or Bic-treated groups was 70% and 14% that of DHT treated group, respectively. On the other side, the percentage of cells with increased activity in 22Rv1 Enz or Bic-treated groups was 100% and 114% that of DHT treated group, respectively (FIG. 16 F). These results suggest that the proportion of cell sensitivity to antiandrogen is a better surrogate of growth response than bulk cell population analysis.

Validation of the System for Detection of Primary PCa Cells Shredding in Patient's Urine or Bladder from the Prostate

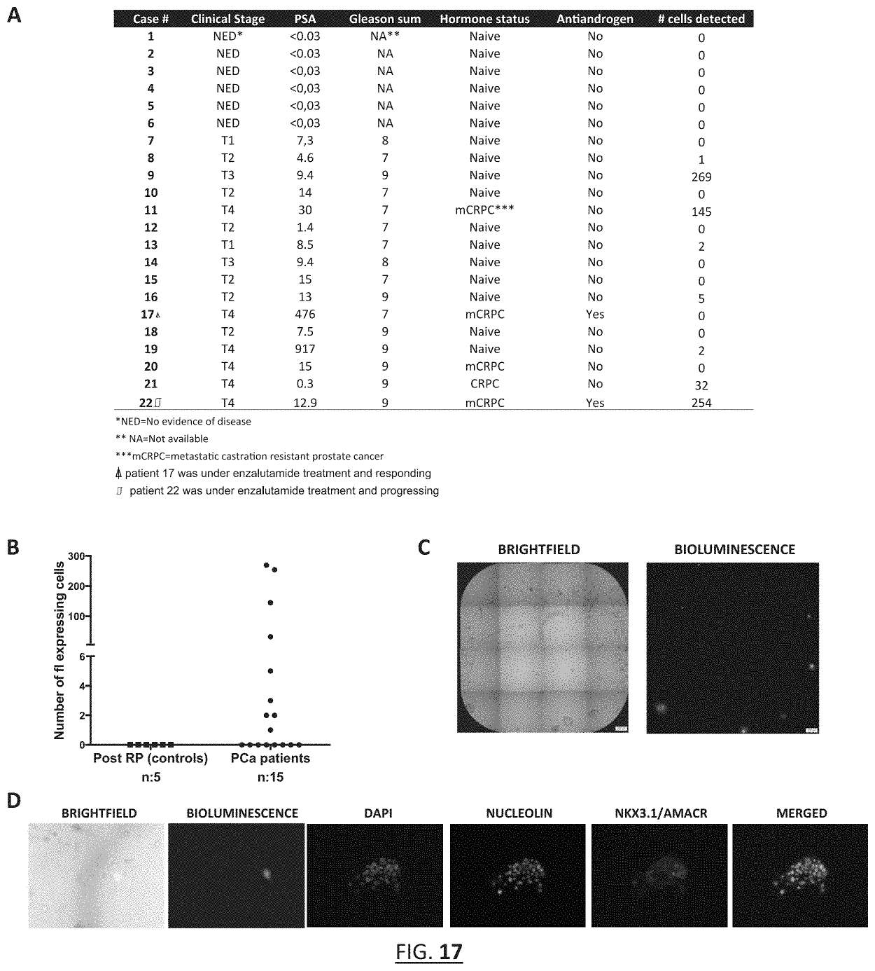

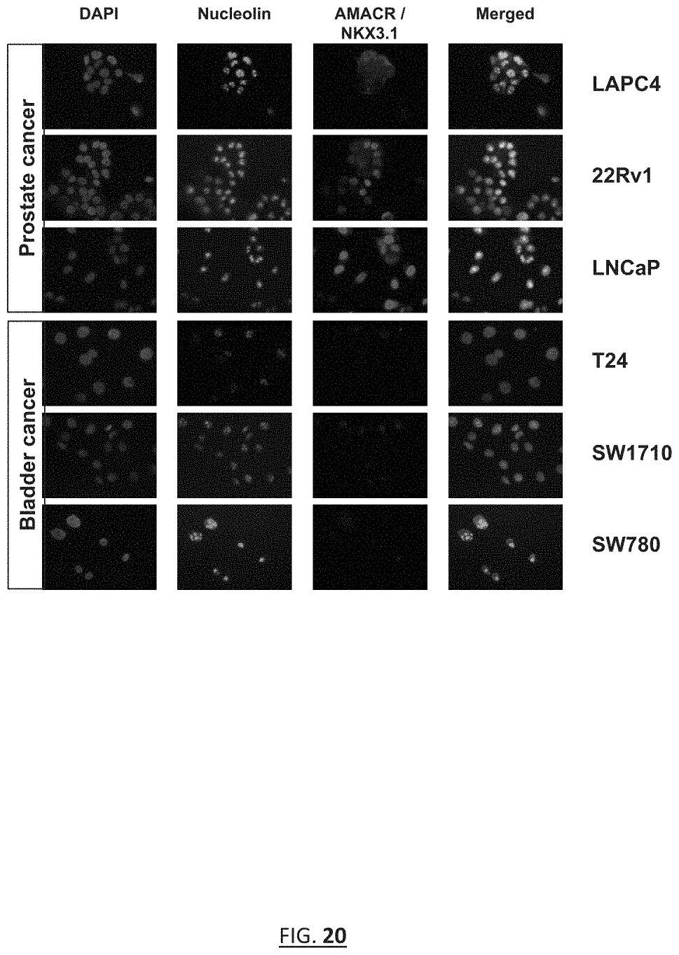

To further validate the system for detection of primary PCa cells shredding in patient's urine or bladder from the prostate, samples were collected from patients in three clinical states; Group 1: post-radical prostatectomy and no evidence of disease; Group 2: with proven localized prostate cancers at transrectal prostate biopsy and Group 3: from castration resistant prostate cancer (CRPC) patients (FIG. 17 A). Isolated cells from samples were transduced with PCA3-Cre-PSEBC-ITSTA and imaged by bioluminescence microscopy. On imaging, bioluminescent cells were detected in none of the Group 1 patients, 62% of Group 2 patients and all patients from Group 3 patients (FIG. 17 B). To ensure luminescent cells were PCa cells as opposed to inflammatory, urothelial or benign cells, positive cells were stained with a panel of markers known to distinguish PCa from non-PCa cells present in the urine (Fujita, 2009). Triple marker immunofluorescence (nucleolin, AMACR and NKX3.1) and DAPI staining after urine cell imaging and fixation showed colocalization of the three signals with luciferase expressing cells. As controls, immunofluorescence for multiple PCa and bladder cancer cell lines showed nucleolin, AMACR and NKX3.1 as a marker panel specific for PCa (FIG. 20). Therefore, based on cell line (FIGS. 1 and 2), control group (FIG. 17 B, Group 1) and immunofluorescence data (FIG. 16 D), it is shown that PCA3-Cre-PSEBC-ITSTA activity is highly specific for the detection of PCa cells (FIG. 16 D).

Dynamic Response to Antiandrogen can be Followed In Vitro and Both CSPC and CRPC Tumors Contain Responding and Non-Responding Cells

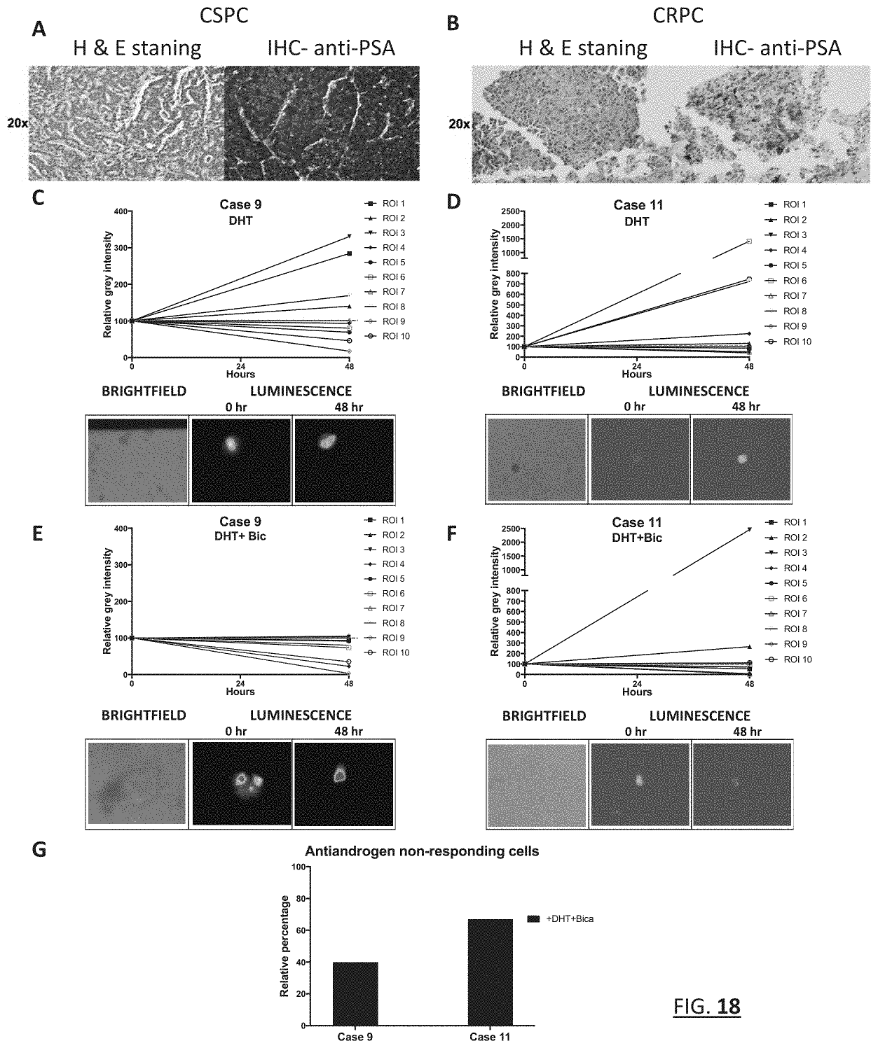

To determine the ability of the system to identify single cell resistant to antiandrogen treatments, urine cells harvested from castration sensitive prostate cancer (CSPC; case 9) and CRPC (case 11) patients were infected with PCA3-Cre-PSEBC-ITSTA in presence of DHT. Seventy-hours after infection, baseline bioluminescence imaging was done (time 0) and the cells were treated with either AR agonist (DHT) or antagonist (Bic). Forty-eight hours later, single cell bioluminescence was measured again. Single cell response to antiandrogen was determined and cells were considered as resistant if luciferase activity increased despite antiandrogen treatment. FIG. 18 A, B shows the IHC from pathological samples harvested from cases 9 and 11 using anti-PSA antibody confirming that endogenous PSA promoter was active in both cases. As observed previously on cell lines, single cell analysis revealed heterogeneous single cell responses after DHT only or with Bic treatment (FIG. 18). On comparing the single cell luciferase expression between the CSPC and CRPC cases, it was found that 60% of cells in the DHT group and 80% and 60% in the DHT+Bic group for patients 9 and 11, respectively, did show a decrease in PSEBC activity (FIG. 18 C). Again, as observed in cell lines, sum of single cell activity changes with time between DHT only or DHT+Bic was not different between CSPC and CRPC patient (data not shown). However, even though most of the cells showed a decrease in luciferase expression due to non-specific cell death and/or response, some cells in each case continued to show an increase in the fl expression revealing a resistant population which could be used as a determinant of treatment resistance. Indeed, similar to cell line analysis (FIG. 16), it was determined whether the percentage of antiandrogen non-responding cells could reflect patient sensitivity. In the CSPC patient (case 9) under Bic treatment, 44% of urine PCa cells had an increase in PSEBC promoter dependent fl activity when compared to DHT only group. This number increased to 67% for the CRPC patient (case 11). These results show that dynamic response to antiandrogen can be followed in vitro and that both CSPC and CRPC tumors contain responding and non-responding cells.

Clinical Follow-Up of a Patient with the System

Finally, in order to correlate the heterogeneity in single PCa cell transcriptional response with clinical response, the clinical follow-up of a third patient (case 22) resistant to Enz at the time of urine PCa cell analysis was analyzed. This patient underwent successful ADT for metastatic PCa in bones for x months. He then developed mCRPC and was treated successfully with Enz for y months after which he developed oligoprogressive disease to the prostate and a single lymph node. Because he presented hematuria and urinary retention, he underwent transurethral resection of the prostate, at which time, bladder cells were harvested for analysis. Because this patient was planned for oligometastasis and prostate radiation therapy within 1 months after TURP, he was placed under Bicalutamide 150 mg because of Enzalutamide resistance. Unfortunately, despite prostate resection, PSA velocity increased after this ENZ resistance unrevealing responding cells despite PSA progression. Also, dynamic bioluminescence microscopy imaging using the PCA3-Cre-PSEBC-ITSTA system on PCa urine cells were performed. The analysis showed that 67% of Enz-treated cells had an increase in PSEBC activity (relative to DHT only treated cells only). Hence, the analysis also revealed that at least a third of cells were sensitive to Enz which correlates with increased PSA velocity after Enzalutamide withdrawal.

PCA3-Cre-PSEBC-ITSTA Allows Dynamic Primary PCa Cell Imaging and Predict Response to Anti-Androgen (Case 17 and Case 22).

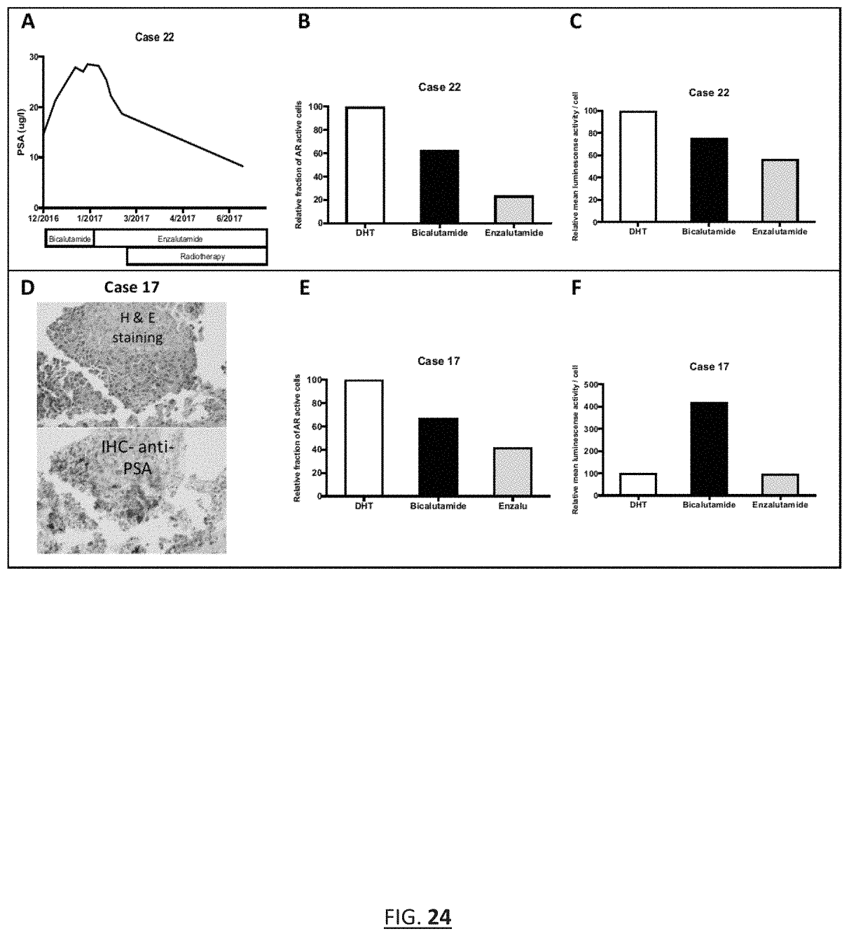

To determine the ability of the system to identify single cell resistant to anti-androgen treatments, urine cells were harvested from castration resistant prostate cancer patients (case 17 and case 22), were infected with PCA3-Cre-PSEBC-ITSTA in presence of DHT. Seventy-hours after infection, baseline bioluminescence imaging was done (time 0) and the cells were treated with either AR agonist (DHT) or antagonist (bicalutamide or enzalutamide). Forty-eight hours post-treatment, single cell bioluminescence was measured again. For case 22, sampling was done as a naïve state before any treatment was received. Similar analysis as cell lines was performed. The ratio of active AR single cell reflected the clinical state of sensitivity of patients to bicalutamide or enzalutamide. Under bicalutamide treatment 63% of urine tumor cells were seen to be AR active, this value decrease to 24% under enzalutamide predicting the patient to be a responder to enzalutamide, corroborating to the clinical PSA follow-up of the patient (FIG. 24). Moreover, the mean luminescence activity per cell is relatively high (56.6%) indicating a population of cells with a potential resistance to enzalutamide (FIG. 24C).

For the case 17 a similar profile as that of case 22 was seen with an exception that the AR active cell under bicalutamide treatment show a very high level of AR activity (418.5%) suggesting a potential agonist effect of bicalutamide in this patient. These results show that fraction of AR active cells along with mean luminescence activity per cell can predict the overall response of a patient to anti-androgen therapy.

Monitoring of Cell Activity Following DHT Treatment.

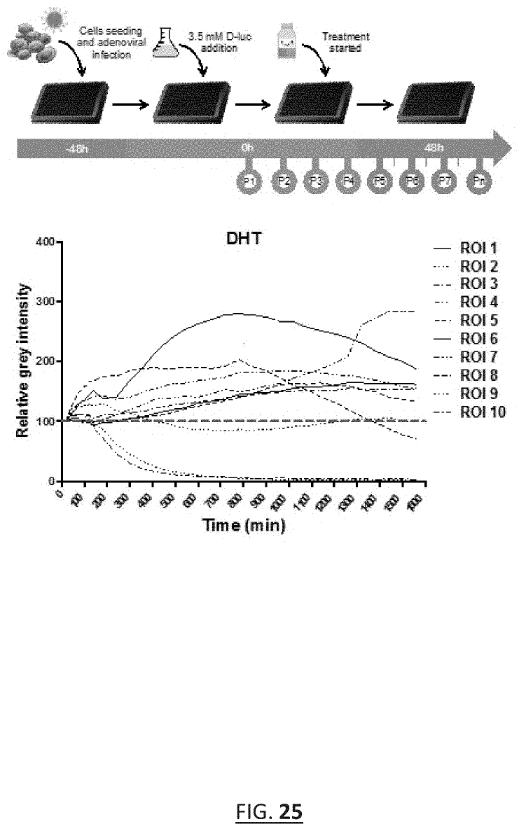

FIG. 25 shows the real time continuous monitoring of cell activity. Sensitivity profiles to drugs could therefore be extracted from single cells or whole cell populations after mathematical modelization.

Material and Method

Isolation and Infection of PCa Cells Isolated from Urine Samples

To test the ability of the PCA3-Cre-PSEBC-TSTA system to detect specifically PCa cells in urine samples were first spiked with 500 22rv1 cells stably expressing m-cherry (kindly provided by Dr. Lily Wu, UCLA) into the urine of a healthy individual. Urine samples were collected in a 50 ml falcon and the cells were spin down at 400 g for 10 mins at 4 degree. Supernatant was carefully removed leaving 2 ml of the samples and not disturbing the pellet too much. Pellet was re-suspended gently and 30 ml of wash buffer (PBS 1×+2% FBS) was added to the pellet and spin again at 200 g for 10 mins at room temperature (RT). Supernatant was again discarded gently without disturbing the pellet and 30 ml of media (RPMI-10% FBS, containing DHT (1 nM), penicillin and streptomycin) was added. Spin was repeated at 200 g for 10 mins at RT. Supernatant was removed carefully leaving behind 400 μl of media containing the isolated cells. 50 μl of media containing the cells was added to each well of a 384 well plate. Isolated cells were infected with either CMV-TSTA or PCA3-Cre-PSEBC-TSTA-fl adenovirus at a concentration of 10⁵ ivp per well. The plate was kept in a shaker overnight. Imaging to detect bioluminescence signal was done using bioluminescence microscopy (LV200). For patients' samples, 10 ml of urine was collected post-DRE (Digital Rectal Examination) from 10 patients having a gleason score of 7 or more and 5 patients post radial prostatectomy as controls.

The above described procedure for isolation was used to isolate cells from these samples. The samples were then infected with 5×10⁵ ivp of PCA3-Cre-PSEBC-TSTA-fl adenovirus. To detect bioluminescence expressing cells and further to determine response to treatment for isolated cells, seventy-two hours after infection, bioluminescence imaging was performed and the treatments were added (vehicle, DHT (1 nM), DHT+Bic (1 nM+10 μM) or DHT+Enz (1 nM+10 μM)). Forty-eight hours later, bioluminescence imaging was repeated on the same cells. Percentage of targeted cells=(number bioluminescence positive cells÷number m-cherry positive cells)×100. The sum of gray intensity values was determined for single cells before and after treatment using CellSens software. Relative gray intensity=((sum gray intensity per ROI−sum gray intensity of background)÷sum gray intensity at 0 h)×100).

Immunofluorescence

22Rv1, LNCaP, LAPC4, DU-145 (prostate cancer cell line) and T24, SW-780, SW1710 (bladder cancer cell line) and 293A (Human embryonic kidney cancer cell line) were seeded in a 384 well plate. Twenty-four hours later media was removed and the cells were fixed by incubating them 50 μl of paraformaldehyde for 20 minutes at RT. Wells were washed with PBS (60 μl per well) three times. After washing the cells were permeabilized using 0.5% triton-X 100 (60 μl per well) for 10 minutes followed by washing with PBS three times. Blocking was then done using PBS-5% BSA (60 μl per well) for 1 hour at RT. The following primary antibodies were diluted in PBS-2% BSA, Anti-AMACR antibody 1:50 (ab93045, Abcam, Toronto, ON, Canada), Anti-Nucleolin antibody 1:200 (ab136649, Abcam, Toronto, ON, Canada), Anti-NKX3.1 1:100 (AES0314, MJSBioLynx Inc., Brockville, ON, Canada), the mix was added to the wells (30 μl per well) and incubated for 1 hour at RT. Washing was done thrice for 5 minutes at RT with PBS containing 0.025% tween20 (60 μl per well). Anti-mouse IgG-Alexa Fluor 488, 1:500 (4408S, New England biolabs) and Anti-Rabbit IgG Alexa Fluor 594, 1:200 (A11012, Thermofisher scientific) were diluted in PBS-2% BSA, added to the wells (30 μl per well) and incubated for 1 hour at RT. Washing was repeated with PBS containing 0.025% tween20 (60 μl per well). Wells were then stained for nuclear counter stain 4-6-diamidino-2-phenylindole (DAPI) for 5 minutes at RT. Washing was done using PBS three times and retained for imaging in distilled water. Imaging for fluorescence done using TURF. For patient samples the cells were fixed after second imaging using paraformaldehyde and the staining was done as described above.

Statistical Analysis

All of the statistical analyses were conducted using the two-sided T-test with Welch's correction, with (*) indicating p≤0.05. The variance was consistent within each experimental group.

Spiked Cell Recovery and Transduction with the PCA3-Cre-PSEBC-ITSTA System into Urine Samples from Healthy Donors

PCa cells (22Rv1 stably expressing mCherry) were spiked in the urine of an healthy individual. Cells were recovered from the sample and transduced with CMV-TSTA or PCA3-Cre-PSEBC-ITSTA in media containing DHT (10 nM). Seventy-two hours after infection, the cells were imaged by adding D-luciferin substrate and with an exposure time of 5 min per image using the bioluminescence microscope.

Single Cell Analysis from Urine Samples with Bioluminescence Microscopy is Representative of the Overall Cell Line Hormonal Responsiveness

Urine samples from healthy patients were spiked with 250 PCa cells stably expressing GFP (22Rv1-GFP or LAPC4-GFP). Urine was spun down to collect all the cells. These cells were infected with 5×10⁵ virus particles of PCA3-Cre-PSEBC-ITSTA. Seventy-two hours after infection imaging was done using the bioluminescence microscope and post-imaging treatments started (DHT (1 nM), DHT (1 nM)+Bic (10 μM) or DHT (1 nM)+Enz (10 μM)). Forty-eight hours post treatment imaging was repeated to assess response on the same cells to determine change in luciferase expression. Relative grey intensity=((sum grey intensity per ROI−sum grey intensity of background)÷sum grey intensity at 0 h)×100).

### REFERENCES

Fujita, K. et al. 2009. Specific Detection of Prostate Cancer Cells in Urine by Multiplex Immunofluorescence Cytology. Human Pathology. 2009, Vol. 40, pp. 924-933.
Goodwin, E. C. & Rottman, F. 1992. The 3′-Flanking Sequence of the Bovine Growth Hormone Gene Contains Novel Elements Required For Efficient and Accurate Polyadenylation. Journal of Biological Chemistry. 1992, Vol. 267, pp. 16330-16334.
Kaczmarczyk, S. J. & Green, J. E. 2001. A Single Vector Containing Modified Cre Recombinase and Lox Recombination Sequences For Inductive Tissue-Specific Amplification of Gene Expression. Nucleic Acids Research. 2001, Vol. 29, e56-e56.
Kwon, Y. & Hecht, N. B. 1993. Binding of a Phosphoprotein to the 3′ Untranslated Region of the Mouse Protamine 2 mRNA Temporally represses its translation. Molecular and Cellular Biology. 1993, Vol. 13, pp. 6547-6557.
Li, H., Xic, N., Gleave, M. E. & Dong, X. 2015. Catalytic Inhibitors of DNA Topoisomerase II Suppress the Androgen Receptor Signaling and Prostate Cancer Progression. Oncotarget. 2015, Vol. 6, pp. 20474-20484.
McNabb, D. S., Reed, R. & Marciniak, R. A. 2005. Dual Luciferase Assay System For Rapid Assessment of Gene Expression in Saccharomyces Cerevisiae. Eukaryotic cell. 2005, Vol. 4, pp. 1539-1549.
Neveu, B. et al. 2016. A PCA3 Gene-based Transcriptional Amplification System Targeting Primary Prostate Cancer. Oncotarget. 2016, Vol. 7, p. 1300.
Pouliot, F., Johnson, M. & Wu, L. 2009. Non-Invasive Molecular Imaging of Prostate Cancer Lymph Node Metastasis. Trends in Molecular Medicine. 2009, Vol. 15, pp. 254-262.
Wu, L. et al. 2001. Chimeric PSA Enhancers Exhibit Augmented Activity in Prostate Cancer Gene Therapy Vectors. Gene Therapy. 2001, Vol. 8, pp. 1416-1426.

1. A method for identifying one or more single cancer cells from cells harvested from a biological fluid of a patient, said method comprising:
providing a bioluminescent substrate;
providing a medium comprising one or more harvested cancer cells transduced with or comprising a construct comprising:
a site specific recombinase under the control of a first system;
a bioluminescent reporter gene under the control of a second system; and
a site-specific recombination sequence, said site-specific recombination sequence located upstream of the bioluminescent reporter gene;
wherein, upon activation of the first and second systems, said transduced cancer cells express the bioluminescent reporter gene following recombination;
wherein at least one of the first and second systems is a response treatment system and the other system is a cancer specific promoter or a tissue specific promoter; and
imaging harvested cancer cells by bioluminescence microscopy to identify said one or more single cancer cells.

2. The method of claim 1, further comprising the step of dynamically assessing the response of the one or more single cancer cells from cells by:
treating the harvested cancer cells with a treatment; and
monitoring the response of the harvested cancer cells by bioluminescence microscopy to determine whether the one or more cancer cells have responded to the treatment.

3. The method of claim 2, wherein the determination of whether the one or more cancer cells have responded to the treatment is based on the difference between a baseline reading and a one or more post-treatment reading.

4. The method of claim 1, wherein the cancer specific promoter is PCA3, PEG3, PEG3AP1, N-Myc or BRN2.

5. The method of claim 2, wherein the cancer specific promoter is PCA3, PEG3, PEG3AP1, N-Myc or BRN2

6. The method of claim 1, wherein the response treatment system is a response treatment promoter.

7. The method of claim 6, wherein the response treatment promoter is the PSA or PSEBC promoter.

8. The method of claim 3, wherein the response treatment system is a response treatment promoter.

9. The method of claim 8, wherein the response treatment promoter is the PSA or PSEBC promoter

10. The method of claim 1, wherein the site-specific recombinase is a recombinase which recognizes cognate site-specific recombination sequence and operates in mammalian cells.

11. The method of claim 10, wherein the site-specific recombinase is Cre recombinase (locus loxP and loxM, element), Flp recombinase (short flippase recognition (FRT) target), øC31 or cas9 CRISPR/Cas9 system.

12. The method of claim 1, wherein the site-specific recombinase is Cre recombinase comprising a BGH intron (e.g. SEQ ID. NO:5).

13. The method of claim 2, wherein the site-specific recombinase is a recombinase which recognizes cognate site-specific recombination sequence and operates in mammalian cells.

14. The method of claim 13, wherein the site-specific recombinase is Cre recombinase (locus loxP and loxM, element), Flp recombinase (short flippase recognition (FRT) target), øC31 or cas9 CRISPR/Cas9 system.

15. The method of claim 2, wherein the site-specific recombinase is Cre recombinase comprising a BGH intron (e.g. SEQ ID. NO:5).

16. The method of claim 1, wherein the biological fluid is blood or urine.

17. The method of claim 2, wherein the biological fluid is blood or urine

18. A construct as defined claim 1.

19. A biological sample of a cancer patient comprising the construct as defined in claim 18 or an isolated primary cancer cell transduced with a construct as defined in claim 18.

20. A method for identifying one or more single prostate cancer cells from cells harvested from the blood or urine of a patient, said method comprising:
providing a bioluminescent substrate;
providing a culture medium comprising one or more harvested cancer cells transduced with a construct comprising:
a Cre recombinase under the control of a PCA3 promoter;
a bioluminescent reporter gene under the control of a PSA promoter; and
a Cre site-specific recombination sequence said Cre site-specific recombination sequence located upstream of the bioluminescent reporter gene;
wherein, upon activation of the PCA3 and PSA promoters, said transduced cancer cells express the bioluminescent reporter gene following recombination; and
imaging harvested prostate cancer cells by bioluminescence microscopy to identifying one or more single prostate cancer cells;
wherein:
the sequence of the PCA3 promoter comprises a nucleic acid at least 70%, 75%, 80%, 85%, 90%, or 95% identical to sequence of SEQ ID NO: 1;
the sequence of the PSA promoter comprises a nucleic acid at least 70%, 75%, 80%, 85%, 90%, or 95% identical to sequence of SEQ ID NO: 2;
the sequence of the Cre recombinase comprises a nucleic acid at least 70%, 75%, 80%, 85%, 90%, or 95% identical to sequence of SEQ ID NO: 4; and
the Cre site-specific recombination sequence is a loxp site comprising a STOP sequence and the STOP sequence comprises a nucleic acid at least 70%, 75%, 80%, 85%, 90%, or 95% identical to sequence of SEQ ID NO: 3.

21. The method of claim 20, further comprising the step of dynamically assessing the antiandrogen response of the one or more single cancer cells to an antiandrogen treatment by:
treating the harvested prostate cancer cells with an antiandrogen treatment; and
monitoring the response of the harvested cancer cells by bioluminescence microscopy to determine whether the one or more cancer cells have responded to the treatment.

22. The method of claim 20, wherein the sequence of the construct comprises a nucleic acid at least 70%, 75%, 80%, 85%, 90%, or 95% identical to sequence of SEQ ID NO: 6.

23. The method of claim 20, wherein said construct comprises an amplification system comprising at least one responsive element sequence and an activator sequence, said amplification system located upstream of the bioluminescent reporter gene.

24. The method of claim 20, wherein said construct comprises an amplification system comprising at least one responsive element sequence and an activator sequence, said amplification system located upstream of the bioluminescent reporter gene and downstream of the second or PSEBC promoter.

25. The method of claim 20, wherein the activator sequence encodes GAL4-VP16 or GAL4-VP2 polypeptide and wherein the responsive element sequence comprises GAL4RE sequence.

26. A construct as defined claim 20.

27. A biological sample of a cancer patient comprising the construct as defined in claim 26 or an isolated primary cancer cell transduced with a construct as defined in claim 26.
